(19) United States
(12) Patent Application Publication
(10) Pub. No.: US 2022/0071653 A1
(43) Pub. Date: Mar. 10, 2022

(54) SYSTEMS AND METHODS FOR DEPOSITING MATERIAL IN A PATIENT

(71) Applicant: Fractyl Laboratories, Inc., Lexington, MA (US)

(72) Inventors: Shweta Mani, Burlington, MA (US); Jason Allen West, Boston, MA (US); Harith Rajagopalan, Wellesley Hills, MA (US); David Maggs, Boston, MA (US); Jay Capalan, Boston, MA (US); Thomas C. Kochem, Watertown, MA (US); Gregory Andrew Dierksen, Brookline, MA (US); Michael Biasella, Medford, MA (US); R. Maxwell Flaherty, Topsfield, MA (US); J. Christopher Flaherty, Auburndale, FL (US)

(73) Assignee: Fractyl Laboratories, Inc., Lexington, MA (US)

(21) Appl. No.: 17/214,157

(22) Filed: Mar. 26, 2021

Related U.S. Application Data
(63) Continuation of application No. PCT/US19/54088, filed on Oct. 1, 2019.
(60) Provisional application No. 62/739,470, filed on Oct. 1, 2018.

(51) Int. Cl. A61B 17/32; A61K 35/38; A61M 5/14; C12N 5/071; G01N 33/50

(52) U.S. Cl. CPC A61B 17/320016; A61K 35/38; A61M 5/14; A61B 2018/00494; G01N 33/5005; A61M 2210/106; C12N 5/0679

(57) ABSTRACT

A system for treating a medical condition comprises a harvesting device, a processing device, and a depositing device. The harvesting device harvests tissue from a mammalian subject at a harvest site. The processing device processes the harvest tissue. The depositing device deposits material in a patient at a deposit site, the material based on the harvested and processed tissue. The deposited material is configured to generate resultant tissue configured to treat the medical condition of the patient. Methods are also provided, the methods including harvesting and processing tissue, and treating a patient by depositing material in the patient, the material being based on the harvested and processed tissue.

### CROSS-REFERENCE TO RELATED APPLICATIONS

This application is a continuation of PCT Application No. PCT/US19/54088 (Attorney Docket No. 41714-718.601, Client Docket No. MCT-037-PCT), filed Sep. 30, 2019, which claims the benefit of and priority to U.S. Provisional Application No. 62/739,470 (Attorney Docket No. 41714-718.101, Client Docket No. MCT-037-PR1), filed Oct. 1, 2018, the contents of which are incorporated herein by reference in its entirety for all purposes.

This application is related to: U.S. patent application Ser. No. 14/515,324 (Attorney Docket No. 41714-705.301, Client Docket No. MCT-003-US), entitled “Tissue Expansion Devices, Systems and Methods”, filed Oct. 15, 2014; U.S. patent application Ser. No. 14/609,332 (Attorney Docket No. 41714-706.301, Client Docket No. MCT-004-US), entitled “Electrical Energy Ablation Systems, Devices and Methods for the Treatment of Tissue”, filed Jan. 29, 2015; U.S. patent application Ser. No. 14/609,334 (Attorney Docket No. 41714-707.301, Client Docket No. MCT-005-US), entitled “Ablation Systems, Devices and Methods for the Treatment of Tissue”, filed Jan. 29, 2015; U.S. patent application Ser. No. 14/673,565 (Attorney Docket No. 41714-708.301, Client Docket No. MCT-009-US), entitled “Methods, Systems and Devices for Performing Multiple Treatments on a Patient”, filed Mar. 30, 2015; U.S. patent application Ser. No. 14/917,243 (Attorney Docket No. 41714-710.301, Client Docket No. MCT-023-US), entitled “Systems, Methods and Devices for Treatment of Target Tissue”, filed Mar. 7, 2016; U.S. patent application Ser. No. 15/274,948 (Attorney Docket No. 41714-712.301, Client Docket No. MCT-027-US), entitled “Injectate Delivery Devices, Systems and Methods”, filed Sep. 23, 2016; U.S. patent application Ser. No. 15/406,572 (Attorney Docket No. 41714-713.301, Client Docket No. MCT-029-US), filed Jan. 13, 2017; U.S. patent application Ser. No. 15/683,713 (Attorney Docket No. 41714-714.301, Client Docket No. MCT-028-US-CIP1-CON1), entitled “Systems, Devices, and Methods for Performing Medical Procedures in the Intestine”, filed Aug. 22, 2017; U.S. patent application Ser. No. 15/812,969 (Attorney Docket No. 41714-714.302, Client Docket No. MCT-028-US-CIP2-CON1), entitled “Systems, Devices, and Methods for Performing Medical Procedures in the Intestine”, filed Nov. 14, 2017; U.S. patent application Ser. No. 15/917,480 (Attorney Docket No. 41714-703.302, Client Docket No. MCT-001-US-CON1), entitled “Devices and Methods for the Treatment of Tissue”, filed Mar. 9, 2018; International PCT Patent Application Number PCT/US2018/042438 (Attorney Docket No. 41714-715.601, Client Docket No. MCT-025-PCT), entitled “Intestinal Catheter Device and System”, filed Jul. 17, 2018; International PCT Patent Application Number PCT/US2019/012338 (Attorney Docket No. 41714-717.601, Client Docket No. MCT-036-PCT), entitled “Material Depositing System for Treating a Patient”, filed Jan. 4, 2019; U.S. patent application Ser. No. 16/267,771 (Attorney Docket No. 41714-711.302, Client Docket No. MCT-024-US-CON1), entitled “Systems, Devices and Methods for the Creation of a Therapeutic Restriction in the Gastrointestinal Tract”, filed Feb. 5, 2019; U.S. patent application Ser. No. 16/379,554 (Attorney Docket No. 41714-709.302, Client Docket No. MCT-013-US-CON1), entitled “Methods, Systems and Devices for Reducing the Luminal Surface Area of the Gastrointestinal Tract”, filed Apr. 9, 2019; U.S. patent application Ser. No. 16/400,491 (Attorney Docket No. 41714-716.301, Client Docket No. MCT-035-US), entitled “Systems, Devices, and Methods for Performing Medical Procedures in the Intestine”, filed May 1, 2019; U.S. patent application Ser. No. 16/438,362 (Attorney Docket No. 41714-704.302, Client Docket No. MCT-002-US-CON1), entitled “Heat Ablation Systems, Devices and Methods for the Treatment of Tissue”, filed Jun. 11, 2019; the contents of each of which is incorporated herein by reference in its entirety for all purposes.

### BACKGROUND OF THE INVENTION

### 1. Field of the Invention

The present invention relates generally to systems for depositing material on and/or in a patient, in particular to systems that harvest tissue, and deposit a material that includes the harvested tissue, in a portion of the patient's gastrointestinal tract.

Bariatric surgeries, such as the Roux-en-Y gastric bypass, have proven their effectiveness in the prevention, treatment, and reversal of a spectrum of cardiovascular, metabolic, and cancer disorders, among others. At least some portion of the metabolic improvement is driven by changes in the hormonal or neuro-hormonal signaling from the intestinal mucosa to the rest of the body, altering insulin production and insulin sensitivity in many organs throughout the body, altering hunger versus satiety, and influencing metabolism in other ways as well. However, bariatric surgeries are invasive, expensive, and carry an important risk of complications. These features limit the scalable use of bariatric surgery to all patients with relevant diseases. There is a need for systems, devices, and methods that deliver a similar metabolic benefit to the surgeries described above but avoid their risks, significant costs, and other limitations.

Disorders of the intestine, such as celiac disease and inflammatory bowel disease, have significant morbidity and mortality for patients around the world. Intestinal procedures in these patients are currently limited to diagnostic procedures, such as mucosal biopsy, or palliative interventional procedures. There is a need for systems, devices, and methods for improved treatment of disorders of the intestine.

### BRIEF SUMMARY OF THE INVENTION

According to one aspect of the present inventive concepts, a system for treating a medical condition, comprising: a harvesting device for harvesting tissue from a mammalian subject at a harvest site; a processing device for processing the harvest tissue; and a depositing device for depositing material in a patient at a deposit site, the material based on the harvested and processed tissue. The deposited material is configured to generate resultant tissue configured to treat the medical condition of the patient.

In some embodiments, the harvesting device is configured to perform an inside-out biopsy.

In some embodiments, the processing device is configured to process the tissue.

In some embodiments, the system further comprises a treatment device for treating tissue. The treatment device can treat tissue at the deposit site and/or tissue proximate the deposit site. The treatment device can treat tissue prior to, during, and/or after deposit of the material at the deposit site. The treatment device can be configured to perform a tissue expansion procedure, and the treatment device can be configured to introduce a fluid into tissue proximate the deposit site. The fluid can comprise saline. The treatment device can be configured to introduce the fluid into the tissue prior to, during, and/or after deposit of the material at the deposit site. The tissue expansion procedure can be performed to enhance distribution of the material at the deposit site. The treatment device can introduce a tissue-expanding fluid comprising a volume of at least 1 mL, 5 mL, 10 mL, 15 mL, or 20 mL. The fluid can include a visualizable agent selected from the group consisting of: an agent visualizable by a visible light camera, such as methylene blue; a fluorescent agent; an agent visualizable by an infrared camera; an agent visualizable by ultrasound; an agent radiographically visualizable; and combinations thereof. The treatment device can include one, two, three, or more fluid delivery elements. The fluid delivery elements can comprise needles. The fluid delivery elements can comprise waterjets. The fluid delivery elements can be configured to deliver an expansion fluid. The fluid delivery elements can be configured to deliver the material. The fluid delivery elements can be configured to deliver both an expansion fluid and the material. The fluid delivery elements can be configured to deliver the expansion fluid and subsequently deliver the material. The treatment device can be configured to expand two or more axial and/or circumferential segments of tissue at the deposit site. The material can be subsequently deposited at the expanded segments.

In some embodiments, the depositing device further comprises a treatment device configured to treat tissue proximate the deposit site. The depositing device can be configured to both treat tissue proximate the depositing device and deliver the material to the deposit site.

In some embodiments, the system further comprises an access device for introducing at least the harvesting device into the mammalian subject.

In some embodiments, the system further comprises an agent configured to be delivered to the patient and/or the mammalian subject.

In some embodiments, the system further comprises a diagnostic kit including one or more components configured to perform an analysis. The diagnostic device can be configured to perform an analysis of a first patient and/or a second patient. The diagnostic device can be configured to perform an analysis of first patient and/or second patient tissue. The diagnostic device can be configured to perform an analysis of the material. The diagnostic kit includes an endoscope, and the endoscope can be configured to screen the patient for cancer, infection, and/or other gastrointestinal pathology. The patient can be excluded from treatment upon the identification of cancer, infection, and/or other gastrointestinal pathology. The diagnostic kit can include components to perform a tissue test on the tissue. The tissue test can be configured to confirm the absence of a characteristic selected from the group consisting of: infected tissue; undesired bacteria; endotoxins and/or other toxins; cancerous tissue; mycoplasma; undesired proteins, such as GIP, GLP-1, other incretins, and/or other secreted proteins, and such as a test including a response to a glucose-based stimulus and/or other nutrient-based stimulus; a virus; undesired bacteria; E. coli; an adventitious agent; other undesirable tissue characteristics; and combinations thereof. The tissue test can be configured to screen for a condition selected from the group consisting of: cancer, such as a cancer of the GI tract; an infection, such as an infection of the GI tract; presence of Clostridium difficile bacteria (C. difficile); HIV; Hepatitis virus A, B, and/or C; syphilis; tuberculosis; and combinations thereof. The tissue test can be configured to confirm the tissue can be from a particular donor. The tissue test can be configured to confirm the presence of a desired material, such as a material selected from the group consisting: desired proteins, such as GIP, GLP-1, other incretins, and/or other secreted proteins, and such as a test including a response to a glucose-based stimulus and/or other nutrient-based stimulus; immune cells; stem cells; enteroendocrine cells; a genetic sequence, such as an mRNA expression; cell surface antigens; and combinations thereof. The tissue test can comprise a donor confirmation test comprising a test selected from the group consisting of: DNA test; mRNA assay; proteomics assay; flow cytometry assay; immunohistochemical analysis; enzyme-linked immunosorbent assay (ELISA); and combinations thereof. The diagnostic kit can be configured to assess a parameter related to an expansion of tissue. The diagnostic kit can be configured to assess a quantity of tissue. The quantity can comprise a quantity of cells. The diagnostic kit can be configured to assess a concentration and/or ratio of one or more substances of tissue. The diagnostic kit can be configured to assess a parameter during and/or after the tissue can be expanded. The diagnostic kit can be configured to confirm an adequate quantity of the material and/or confirm other expansion parameters selected from the group consisting of: cell growth rate; organoid growth rate; organoid density, such as within a growth substrate; morphometry of organoids, including a quantification of the number of buds and/or crypts in the organoids; cell culture media secretions, such as GIP, GLP-1, insulin, and/or other marker peptide not normally secreted by this cell type; and combinations thereof. The diagnostic kit can include components configured to assess a parameter selected from the group consisting of: number of crypts in a tissue sample; basement membrane matrix seeding density, such as derived from crypt count; presence and/or concentration of immune cells; fraction of Lgr5+ cells relative to other cell types; spatial distribution of cells, such as distribution of cells in an organoid, such as distribution of stem cells at the ends of crypts buds, Paneth cells immediately adjacent to Lgr5+ stem cells, and differentiated cells clustered near the central cystic area of an organoid; and combinations thereof. The diagnostic kit can be configured to perform one or more tests to determine successful modification of cells in tissue. The one or more tests can be selected from the group consisting of: PCR-based test; reporter proteins test, such as an eGFP test; his-tagging test, such as a reporter protein and/or of an otherwise functional protein of interest; antibiotic selection test, such as a test for resistance to puromycin; a test for modified surface and/or transmembrane proteins; and combinations thereof. The diagnostic kit can be configured to perform one or more tests on byproducts produced during the processing of the tissue and/or the material. The diagnostic kit can be configured to perform a test to determine safety and/or efficacy of the material prior to deposit in the patient. The diagnostic kit can be configured to confirm a parameter selected from the group consisting of: endotoxin and/or other toxin levels are below a threshold; bioburden is below a threshold; mycoplasma level below a threshold; adventitious agent below a threshold; cell viability above a threshold, such as above a threshold of 70%; percentage of Lgr5+ cells above a threshold, such as above a threshold of 1%, 2%, or 30%; percentage of Paneth cells above a threshold, such as above a threshold of 1%, 2%, or 30%; transduction copies per cell above threshold; potency above a threshold; and combinations thereof. The diagnostic kit can be configured to assess potency of the material. The potency assessment can comprise a quantified expression of incretins and/or an expression ratio of one incretin to another. The potency assessment can comprise expressions compared to a threshold to determine adequacy of the material. The potency assessment can comprise a quantification of the number of cells, crypts, and/or organoids in the material. The diagnostic kit can be configured to provide identity information of the material. The information can comprise anatomical location information. The diagnostic kit can be configured to quantify and/or detect markers of source tissue from cells other than enteroendocrine cells. The diagnostic kit can be configured to perform a blood test, such as a blood test of a first patient and/or a second patient. The blood test can evaluate the patient's suitableness for the treatment. The blood test can evaluate circulating levels of hormones, presence of particular antibodies, and/or blood borne markers.

In some embodiments, the system further comprises a storage kit including one or more components configured to store the tissue and/or material. The storage kit can comprise one or more containers that includes a unique identification. The storage kit can comprise one or more environmentally controlled containers. The storage kit can include one or more cleaning and/or other agents to wash the tissue and/or material. The storage kit can comprise a washing agent selected from the group consisting of: surfactant; detergent, such as Triton X-100 or SDS; buffered saline; and combinations thereof. The storage kit can include one or more storage solutions. The storage solution can comprise a preservative selected from the group consisting of: a preservative configured to arrest cellular apoptosis; a Rho kinase inhibitor, such as Y27632, and such as at a concentration of 5 uM to 15 uM; an antimicrobial reagent, such as Primocin, and such as at a 0.1% to 0.3% v/v solution; dimethyl sulfoxide (DMSO), such as at a 10% v/v solution, and such as for cryopreserved shipment; and combinations thereof.

In some embodiments, the system further comprises a safety assembly including one or more components configured to assure the safety and/or efficacy of the material prior to its deposit in the patient. The safety assembly can be configured to confirm the tissue and/or material can be not exposed to an undesired temperature, such as exposure to high or low temperatures for an undesired amount of time. The safety assembly can be configured to confirm the tissue and/or material can be not exposed to an undesired pressure. The safety assembly can be configured to confirm the tissue and/or material can be not exposed to an undesired force. The safety assembly can be configured to confirm the tissue and/or material can be not exposed to an undesired pH level. The safety assembly can be configured to confirm the tissue and/or material can be not exposed to an undesired physical state, and the safety assembly can comprise one or more sensors selected from the group consisting of: temperature; pressure; force; pH; and combinations thereof. The one or more sensors can be configured to accompany the tissue and/or material during storage and/or transportation. The safety assembly can comprise one or more components configured to destroy the material if an adverse condition is detected. The safety assembly includes a portion of a tissue modification kit, the portion comprising genetic material incorporated into the material, and the genetic material can be configured to cause death of a cell within the material when the adverse condition is detected.

In some embodiments, the system further comprises an identification kit including one or more components configured to identify the tissue and/or material prior its deposit in the patient.

In some embodiments, the system further comprises a tissue expansion kit including one or more components configured to culture, amplify, and/or otherwise expand the harvest tissue prior to its deposit in the patient. The tissue expansion kit can comprise tissue culture medium. The tissue culture medium can comprise basal medium. The tissue culture medium can comprise growth medium selected from the group consisting of: DMEM/F12; 10 mM HEPES; 2 mM GlutaMax; 1% PenStrep; and combinations thereof. The tissue culture medium can comprise an additive selected from the group consisting of: a tropic factor; a temporary additive; and combinations thereof. The tissue expansion kit can comprise a growth factor selected from the group consisting of: Gastrin 1, such as at a concentration of 10 nM; N Acetylcysteine (NAC), such as at a concentration of 1 mM; B27 supplement, such as at a concentration of 2% v/v; Wnt3A, such as at a concentration of 100 ng/mL; R-spondin 1, such as at a concentration of 1 μg/mL; Noggin, such as at a concentration of 100 ng/mL; epidermal growth factor (EGF), such as at a concentration of 50 ng/mL; A83-01, such as at a concentration of 500 nM; SB202190 (p38 MAP kinase); and combinations thereof. The tissue expansion kit can comprise a temporary additive, such as Y-27632 (ROCK inhibitor) and/or CHIR99021 (GSK-3 inhibitor), and the additive can be added to the tissue at the onset of culturing and then removed after a limited time period. The tissue expansion kit can comprise a putative growth factor. The putative growth factor can include a native molecule. The putative growth factor can include an analog of a substance selected from the group consisting of: insulin; gastrin; betacellulin; amphiregulin; TGF-alpha: transforming growth factor-alpha; epidermal growth factor (EGF); heparin binding epidermal growth factor (HB-EGF); GLP-1: GLP-2; growth hormone; insulin-like growth factor-1 (IGF-1); granulocyte colony stimulating factor (G-CSF); erythropoietin (EPO); intestinal trefoil factor (ITF); keratinocyte growth factor (KGF); hepatocyte growth factor (HGF); neuregulin-4 (NRG-4); and combinations thereof. The tissue expansion kit can include one or more support structures configured to support organoid growth of the material. The support structure can be selected from the group consisting of: a basement membrane matrix comprising a gelatinous protein mixture, such as Matrigel; basement membrane extracts (BMEs); a PEGylated hydrogel; a hydrogel with tunable elasticity, such to provide beneficial effects on cell proliferation and differentiation; and combinations thereof.

In some embodiments, the system further comprises a tissue modification kit including one or more components configured to modify the tissue and/or material prior to its deposit in the patient. The tissue modification kit can be configured to genetically modify the tissue and/or material. The tissue modification kit can comprise a gene delivery medium comprising a mechanism selected from the group consisting of: transposon, such as a PiggyBac transposon; viral vector, such as retrovirus, lentivirus, adenovirus, or adeno-associated virus; CRISPR-Cas9; electroporation and/or sonoporation mechanism; Lipofection; and combinations thereof. The tissue modification kit can be configured to perform a genetic modification selected from the group consisting of: gene “knock-out”, such that a gene can be made inoperative; gene “knock-in”, such that a gene or portion thereof can be substituted for another gene or portion thereof modification of noncoding portions of the genome, such as promoters; insertion of novel genes, such as native or non-native genes; insertion of promoters; insertion of DNA; and combinations thereof. The tissue modification kit includes a piece of genetic material configured to cause cell death when a specific nutrient can be delivered to the cell. The tissue modification kit includes a piece of genetic material configured to cause cell death when the cell can be denied a specific nutrient.

In some embodiments, the system further comprises a cell sorting kit including one or more equipment, materials, and/or components configured to sort cells of the tissue and/or material prior to its deposit in the patient. The cell sorting kit can be configured to perform a cell sorting process using one or more processes selected from the group consisting of: presence of cell surface antigens, such as Lgr5, and such as coupled with FACS and/or MACS; detection of proteins via intracellular staining coupled with flow cytometry, such as when only cells with intracellular GIP can be selected; selection by application of an antibiotic such as puromycin, such as when the transduced cells have antibiotic resistance imparted as part of a genetic modification that has been performed; density gradient separation, such as centrifugation, and such as following a dissociation and/or trypsinization step; filtering through a porous membrane with a particular pore size, such as an approximately 70 micron pore size, and such as following a dissociation and/or trypsinization step; and combinations thereof.

In some embodiments, the system can further comprise a deposit material assembly kit including one or more equipment, materials, containers, and/or components configured to assemble the material prior to its deposit in the patient. The deposit material assembly kit can include one or more preservatives, such as cryopreservative. The deposit material assembly kit can include one or more antibiotics, such as penicillin and/or puromycin. The material can be genetically modified to be antibiotic resistant and the antibiotic can be added to the material to provide an inherent growth advantage over native cells at the deposit site, and the native cells can be not antibiotic resistant.

In some embodiments, the system further comprises an expansion device configured to deliver injectate into tissue at the harvesting and/or deposit site.

In some embodiments, the system further comprises a first treatment device configured to ablate and/or remove tissue at or otherwise proximate the deposit site. The system can further comprise a second treatment device configured to perform a tissue expansion procedure at or otherwise proximate the deposit site. The first treatment device and the second treatment device can be the same device. The depositing device, the first treatment device, and the second treatment device can be the same device.

In some embodiments, the system is configured to treat at least one, at least two, and/or at least three medical conditions selected from the group consisting of: Type 2 diabetes; Type 1 diabetes; Double diabetes; gestational diabetes; hyperglycemia; pre-diabetes; monogenic diabetes; maturity onset diabetes of the young; impaired glucose tolerance; insulin resistance; hyperinsulinemia; hypoinsulinemia; non-diabetic hypoglycemia; elevated albuminuria; non-alcoholic fatty liver disease; non-alcoholic steatohepatitis; obesity; obesity-related disorder; polycystic ovarian syndrome; hypertriglyceridemia; hypercholesterolemia; psoriasis; gastroesophageal reflux disease; coronary artery disease; stroke; transient ischemic attack; cognitive decline; dementia; Alzheimer's Disease; neuropathy; diabetic nephropathy; retinopathy; heart disease; diabetic heart disease; heart failure; diabetic heart failure; hirsutism; hyperandrogenism; fertility issues; menstrual dysfunction; cancer; liver cancer; ovarian cancer; breast cancer; endometrial cancer; cholangiocarcinoma; adenocarcinoma; glandular tissue tumor; stomach cancer; colorectal cancer; prostate cancer; diastolic dysfunction; hypertension; myocardial infarction; microvascular disease related to diabetes; anorexia nervosa; anorexia; a binge eating disorder; a hyperphagic state; hyperphagia; polyphagia; Prader Willi syndrome; an obesity-related genetic disorder; hypoglycemia; hypoglycemia that presents after a bariatric procedure (referred to as “post-bariatric hypoglycemia”); recurrent obesity post-bariatric surgery; recurrent metabolic disease post-bariatric surgery; iron overload conditions such as hemochromatosis types 1-4 and/or bantu siderosis; pancreatic cancer; short bowel syndrome; sleep apnea; arthritis; rheumatoid arthritis; general lipodystrophy (e.g. congenital, Berardinelli-Seip syndrome, acquired, and/or Lawrence syndrome); familial or acquired partial lipodystrophy (e.g. Barraquer-Simons syndrome and/or Köbberling-Dunnigan syndrome); congenital leptin deficiency; lipoprotein lipase deficiency (e.g. familial chylomicronemia syndrome, chylomicronemia, chylomicronemia syndrome, and/or hyperlipoproteinemia type Ia); Hemophilia A; Hemophilia B; Gaucher's disease; Fabry disease; Alpha-1 antitrypsin deficiency; galactosemia (e.g. type 1, 2, and/or 3); Menkes disease; Wilson's disease; microvillus inclusion disease; congenital tufting enteropathy; chronic gastroparesis; eosinophilic intestinal disease; cystic fibrosis; Crohn's disease, inflammatory bowel disease (IBD); eosinophilic esophagitis; Celiac disease; familial apolipoprotein E deficiency; aromatase deficiency; and combinations thereof.

### BRIEF DESCRIPTION OF THE DRAWINGS

FIG. 1 illustrates a system for depositing material at a deposit site of a patient, consistent with the present inventive concepts.

FIG. 2 is a flow chart of a method for depositing material at a deposit site of a patient, consistent with the present inventive concepts.

FIGS. 3A-3C are side sectional anatomic views of steps of a method of performing an inside-out biopsy, consistent with the present inventive concepts.

FIGS. 3D-3E are side sectional anatomic views of two methods of performing an outside-in biopsy, consistent with the present inventive concepts.

### DETAILED DESCRIPTION OF THE INVENTION

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one, two, or more of these.

As used herein, the term “proximate” shall include locations relatively close to, on, in and/or within a referenced component, anatomical location, or other location. As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values and/or outside a threshold range of values, to cause a desired effect (e.g. efficacious therapy) and/or to prevent or at least reduce the effects of (hereinafter “prevent” or “reduce”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. “Positive pressure” includes pressure above room pressure or simply a pressure that is greater than another pressure, such as a positive differential pressure across a fluid pathway component such as a valve. “Negative pressure” includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function. In some embodiments, a functional element comprises one or more elements constructed and arranged to perform a function selected from the group consisting of: deliver energy; extract energy (e.g. to cool a component); deliver a pharmaceutical drug or other agent; manipulate a system component or patient tissue; record or otherwise sense a parameter such as a patient physiologic parameter or a patient anatomical parameter; and combinations of one or more of these. A functional element can comprise a fluid, such as an ablative fluid (as described hereabove) comprising a liquid or gas configured to ablate or otherwise treat tissue. A functional element can comprise a reservoir, such as an expandable balloon configured to receive an ablative fluid. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, thermal energy such as heat energy and/or cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one, two, or more of these.

As used herein, the term “fluid” can refer to a liquid, gas, gel, and/or any flowable material, such as a material which can be propelled through a lumen and/or opening.

As used herein, the term “tissue modification procedure” refers to a procedure performed on tissue to modify a property of the tissue treated and/or tissue proximate the tissue treated (“treated tissue” herein). A tissue modification procedure can result in necrosis and/or removal of tissue, after which “replacement tissue” develops in the place of the removed tissue, the replacement tissue having different properties than the tissue that was removed. A tissue modification procedure can result in a reduction in surface area of the treated tissue (e.g. a reduction in the luminal surface area of an inner wall of tubular tissue), such as to modify secretions and/or absorptions of the tissue. A tissue modification procedure can include: delivery of energy to tissue (e.g. delivery of ablative heat, ablative cold, and/or ablative electromagnetic energy); mechanical removal and/or disruption of tissue; chemical ablation of tissue; and combinations of one, two, or more of these. Treated tissue or replacement tissue (“treated tissue” herein) can have modified properties including but not limited to: modification of one or more absorptive properties of the tissue; modification of one or more secretive properties of the tissue; modification of neuronal signaling of the tissue; and combinations of one, two or more of these. Effects of the tissue modification procedure can occur acutely and/or it can take place over time, such as days, weeks or months. In some embodiments, a tissue modification procedure includes injecting one or more materials into the submucosal tissue of a GI lumen, such as to expand a submucosal tissue layer, for example a full (360°) or partial circumferential expansion of an axial segment of the GI tract. These tissue expansion procedures can cause a relatively acute result (e.g. less than 30 minutes), such as to perform a subsequent mucosal ablation procedure wherein the expanded tissue acts as a safety margin of tissue during the ablation, protecting the underlying layers from damage. In some embodiments, the full or partial circumferential submucosal tissue expansion is performed to cause luminal narrowing at the axial segment, such as is described in applicant's co-pending application U.S. patent application Ser. No. 16/267,771 (Attorney Docket No. 41714-711.302, Client Docket No. MCT-024-US-CON1), entitled “Systems, Devices and Methods for the Creation of a Therapeutic Restriction in the Gastrointestinal Tract”, filed Feb. 5, 2019, the contents of which are incorporated herein by reference in its entirety. This luminal narrowing can be configured to last for a prolonged period of time, such as at least 1 day, at least 1 week, and/or at least one month. This luminal narrowing can be performed to restrict food intake of the patient, and/or for other purposes.

As used herein, the term “ablative temperature” refers to a temperature at which tissue necrosis or other desired tissue treatment occurs (e.g. a temperature sufficiently hot or sufficiently cold to cause tissue necrosis or any desired effect). As used herein, the term “ablative fluid” refers to one or more liquids, gases, gels or other fluids whose thermal properties cause tissue necrosis and/or another desired tissue treatment (e.g. one or more fluids at an ablative temperature). Alternatively or additionally, “ablative fluid” refers to one or more fluids whose chemical properties (at room temperature, body temperature or otherwise) cause tissue necrosis or another desired tissue treatment. A treatment element (e.g. a functional element) of the present inventive concepts can comprise one or more ablative fluids and/or comprise one or more elements that deliver one or more ablative fluids (e.g. deliver the fluids onto a tissue surface and/or into a volume of tissue).

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems and methods for implanting or otherwise depositing material at a deposit site of a patient (e.g. a mammalian patient), such as to provide a therapeutic benefit to the patient. The systems and methods can be configured to treat a medical condition of the patient, such as one, two, three or more diseases, disorders, and/or other medical conditions of a patient. The system includes a depositing device for depositing material at a deposit site, such as material that is based on tissue harvested at a harvest site with a harvesting device of the system. The deposited material generates tissue that is configured to treat the medical condition of the patient.

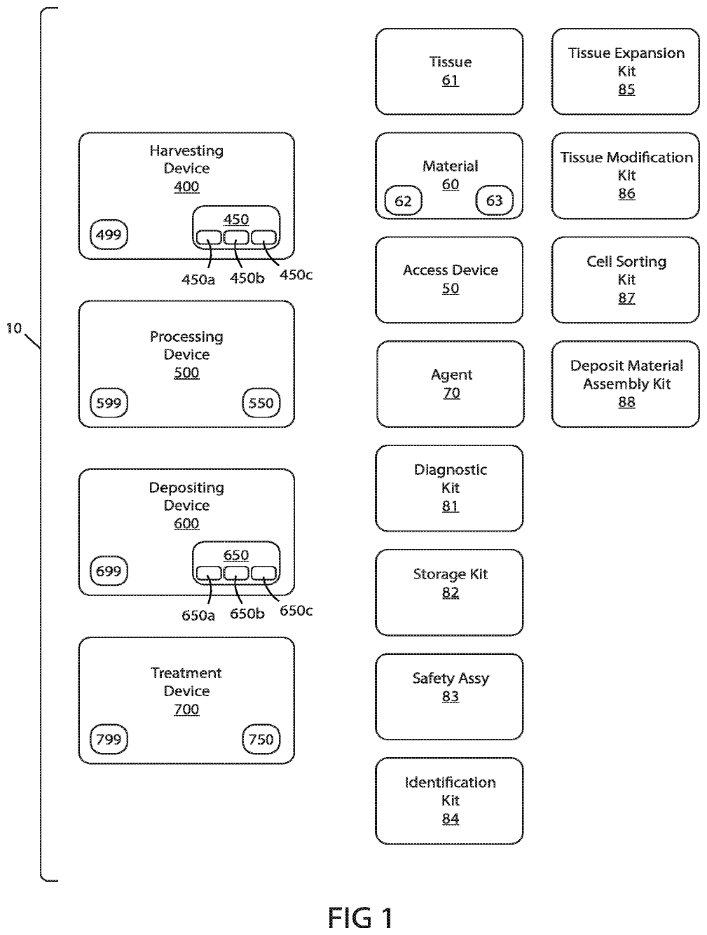

Referring now to FIG. 1, a schematic view of a system for depositing a material at a deposit site of a patient is illustrated, consistent with the present inventive concepts. System 10 includes depositing device 600. Depositing device 600 comprises a device for implanting, placing, seeding, inserting, spraying, topically applying, and/or otherwise depositing (“depositing” herein) material, such as material 60 described herebelow, at a “deposit site” of a patient. The deposit site can comprise one, two, or more sites on and/or within a patient (e.g. on the patient's skin and/or within the body of the patient, respectively). After the depositing of material 60, new tissue is generated at the deposit site and locations proximate the deposit site. The new tissue (including material 60 and/or void of material 60) comprises “resultant tissue” herein. Properties of the resultant tissue can be driven by or otherwise based on material 60 (e.g. properties including one or more proteins that are expressed by the resultant material). Generation of the resultant tissue by the systems and methods of the present inventive concepts can provide a therapeutic benefit used to treat one or more medical conditions of the patient.

In some embodiments, system 10 includes a device for harvesting tissue of the patient, harvesting device 400, which harvests tissue, tissue 61 described herebelow, at a “harvest site” of the patient. The harvest site can include one, two, or more patient tissue locations such as one or more locations on the skin of the patient, and/or one or more locations within the patient's body. In some embodiments, system 10 includes a device, processing device 500, that is configured to process tissue 61. Processing device 500 can be used to process tissue 61 after its harvest by harvesting device 400. In some embodiments, processing device 500 is configured to treat in-situ tissue prior to its harvest. In some embodiments, system 10 includes treatment device 700. Treatment device 700 is configured to treat tissue at a “treatment site” of the patient. The treatment site can include one or more locations on the skin of the patient, and/or one or more locations within the patient's body. In some embodiments, treatment device 700 treats tissue of a deposit site and/or tissue proximate a deposit site, such as when performing a tissue treatment procedure prior to the depositing of material 60 at a deposit site.

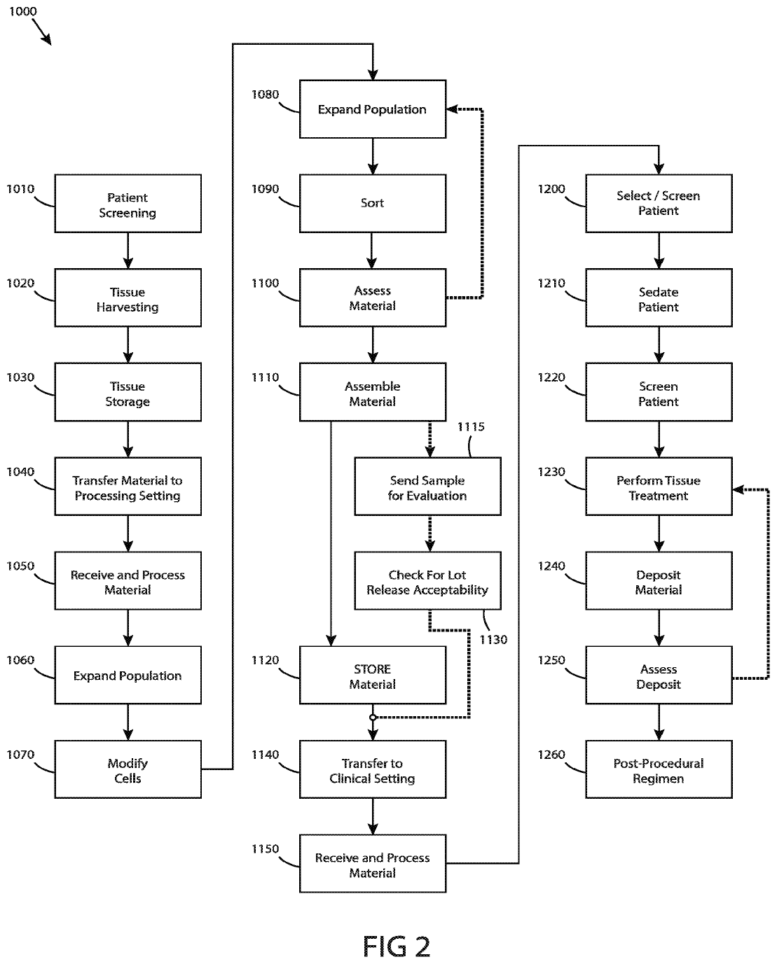

In some embodiments, system 10 is configured to provide a treatment as described herebelow in reference to FIGS. 2 and/or 3. System 10 can be configured to treat one, two, three, or more medical conditions selected from the group consisting of: Type 2 diabetes; Type 1 diabetes; Double diabetes; gestational diabetes; hyperglycemia; pre-diabetes; monogenic diabetes; maturity onset diabetes of the young; impaired glucose tolerance; insulin resistance; hyperinsulinemia; hypoinsulinemia; non-diabetic hypoglycemia; elevated albuminuria; non-alcoholic fatty liver disease; non-alcoholic steatohepatitis; obesity; obesity-related disorder; polycystic ovarian syndrome; hypertriglyceridemia; hypercholesterolemia; psoriasis; gastroesophageal reflux disease; coronary artery disease; stroke; transient ischemic attack; cognitive decline; dementia; Alzheimer's Disease; neuropathy; diabetic nephropathy; retinopathy; heart disease; diabetic heart disease; heart failure; diabetic heart failure; hirsutism; hyperandrogenism; fertility issues; menstrual dysfunction; cancer; liver cancer; ovarian cancer; breast cancer; endometrial cancer; cholangiocarcinoma; adenocarcinoma; glandular tissue tumor; stomach cancer; colorectal cancer; prostate cancer; diastolic dysfunction; hypertension; myocardial infarction; microvascular disease related to diabetes; anorexia nervosa; anorexia; a binge eating disorder; a hyperphagic state; hyperphagia; polyphagia; Prader Willi syndrome; an obesity-related genetic disorder; hypoglycemia; hypoglycemia that presents after a bariatric procedure (referred to as “post-bariatric hypoglycemia”); recurrent obesity post-bariatric surgery; recurrent metabolic disease post-bariatric surgery; iron overload conditions such as hemochromatosis types 1-4 and/or bantu siderosis; pancreatic cancer; short bowel syndrome; sleep apnea; arthritis; rheumatoid arthritis; general lipodystrophy (e.g. congenital, Berardinelli-Seip syndrome, acquired, and/or Lawrence syndrome); familial or acquired partial lipodystrophy (e.g. Barraquer-Simons syndrome and/or Köbberling-Dunnigan syndrome); congenital leptin deficiency; lipoprotein lipase deficiency (e.g. familial chylomicronemia syndrome, chylomicronemia, chylomicronemia syndrome, and/or hyperlipoproteinemia type 1a); Hemophilia A; Hemophilia B; Gaucher's disease; Fabry disease; Alpha-1 antitrypsin deficiency; galactosemia (e.g. type 1, 2, and/or 3); Menkes disease; Wilson's disease; microvillus inclusion disease; congenital tufting enteropathy; chronic gastroparesis; eosinophilic intestinal disease; cystic fibrosis; Crohn's disease, inflammatory bowel disease (IBD); eosinophilic esophagitis; Celiac disease; familial apolipoprotein E deficiency; aromatase deficiency; and combinations of these. In some embodiments, system 10 is configured to treat at least two, or at least three of the above medical conditions.

As used herebelow and otherwise herein, “producing a substance X” and its derivatives shall include: producing substance X; producing an analog of substance X; producing an agonist of substance X; activating production of substance X; activation of a receptor of substance X; and/or activating downstream signaling pathways that are triggered via the activation of the receptor for substance X. Producing substance X can include constitutive production of substance X or regulated production of substance X, such as nutrient-responsive production and/or secretion of substance X.

As used herebelow and otherwise herein, “reducing a substance X” and its derivatives shall include: removing or at least reducing substance X; sequestering substance X; providing agents that counteract substance X; sequestering, reducing, or eliminating the production of substance X; inhibiting or blocking a receptor of substance X; and/or reducing or inhibiting downstream signaling pathways influenced by a receptor of substance X.

As used herebelow and otherwise herein, the term “gene disruption” and its derivatives refer to a procedure which eliminates or at least reduces the production of functional gene products from one, both, or multiple alleles of the gene. Gene disruptions can be produced by specific targeting of a gene locus via DNA editing technologies such as CRISPR/Cas9, Zinc-finger nucleases, or TALENs, which have both DNA-binding activity that allow targeting of a specific DNA sequence or sequences as well as DNA-cleaving activity that introduces double-stranded DNA breaks that are then repaired predominantly by non-homologous end joining (NHEJ). NHEJ can lead to imprecise DNA repair whose mutations lead to effective gene disruption.

In some embodiments, the patient has Type 2 diabetes, and a treatment of the present inventive concepts can be performed to modify mucosa (e.g. duodenal mucosa) in order to achieve one or more of the following effects: produce insulin, such as a nutrient responsive production of insulin; produce GLP-1, such as constitutive and/or nutrient-responsive GLP-1 and/or PYY; and/or reduce GIP (e.g. to reduce GIP production and/or to produce a GIP antagonist, as defined herein).

In some embodiments, the patient has Type 1 diabetes, and a treatment of the present inventive concepts can be performed to modify mucosa (e.g. duodenal mucosa) in order to achieve one or more of the following effects: produce nutrient responsive insulin; and/or disrupt the FOX01 gene.

In some embodiments, the patient has double diabetes, and a treatment of the present inventive concepts can be performed to modify mucosa (e.g. duodenal mucosa) in order to achieve one or more of the following effects: produce insulin, such as nutrient responsive production of insulin; produce GLP-1, such as constitutive and/or nutrient-responsive GLP-1 and/or PYY; and/or reduce GIP.

In some embodiments, the patient has gestational diabetes, and a treatment of the present inventive concepts can be performed to modify mucosa (e.g. duodenal mucosa) in order to achieve one or more of the following effects: produce insulin, such as nutrient responsive production of insulin; produce GLP-1, such as constitutive and/or nutrient-responsive GLP-1 and/or PYY; and/or reduce GIP.

In some embodiments, the patient has hyperglycemia, and a treatment of the present inventive concepts can be performed to modify mucosa (e.g. duodenal mucosa) in order to achieve one or more of the following effects: produce insulin, such as nutrient responsive production of insulin; produce GLP-1, such as constitutive and/or nutrient-responsive GLP-1 and/or PYY; and/or reduce GIP.

In some embodiments, the patient has pre-diabetes, and a treatment of the present inventive concepts can be performed to modify mucosa (e.g. duodenal mucosa) in order to achieve one or more of the following effects: produce insulin, such as nutrient responsive production of insulin; produce GLP-1, such as constitutive and/or nutrient-responsive GLP-1 and/or PYY; and/or reduce GIP.

In some embodiments, the patient has monogenic diabetes, and a treatment of the present inventive concepts can be performed to modify mucosa (e.g. duodenal mucosa) in order to achieve one or more of the following effects: produce insulin, such as nutrient responsive production of insulin; produce GLP-1, such as constitutive and/or nutrient-responsive GLP-1 and/or PYY; and/or reduce GIP.

In some embodiments, the patient has maturity onset diabetes of the young, and a treatment of the present inventive concepts can be performed to modify mucosa (e.g. duodenal mucosa) in order to achieve one or more of the following effects: produce insulin, such as nutrient responsive production of insulin; produce GLP-1, such as constitutive and/or nutrient-responsive GLP-1 and/or PYY; and/or reduce GIP.

In some embodiments, the patient has impaired glucose tolerance, and a treatment of the present inventive concepts can be performed to modify mucosa (e.g. duodenal mucosa) in order to achieve one or more of the following effects: produce insulin, such as nutrient responsive production of insulin; produce GLP-1, such as constitutive and/or nutrient-responsive GLP-land/or PYY; and/or reduce GIP.

In some embodiments, the patient has insulin resistance, and a treatment of the present inventive concepts can be performed to modify mucosa (e.g. duodenal mucosa) in order to achieve the following effect: reduce GIP.

In some embodiments, the patient has hyperinsulinemia, and a treatment of the present inventive concepts can be performed to modify mucosa (e.g. duodenal mucosa) in order to achieve one or more the following effects: produce GLP-1, such as constitutive and/or nutrient-responsive GLP-1 and/or PYY; and/or Leptin and/or Amylin; and/or reduce GIP.

In some embodiments, the patient has hypoinsulinemia, and a treatment of the present inventive concepts can be performed to modify mucosa (e.g. duodenal mucosa) in order to achieve one or more of the following effects: produce insulin, such as nutrient responsive production of insulin; and/or produce GLP-1, such as constitutive and/or nutrient-responsive GLP-1 and/or PYY.

In some embodiments, the patient has non-diabetic hypoglycemia, and a treatment of the present inventive concepts can be performed to modify mucosa (e.g. duodenal mucosa) in order to achieve the following effect: produce glucagon, such as a production of glucagon in response to circulating insulin and/or circulating c-peptide.

In some embodiments, the patient has elevated albuminuria, and a treatment of the present inventive concepts can be performed to modify mucosa (e.g. duodenal mucosa) in order to achieve one or more of the following effects: produce insulin, such as a nutrient responsive production of insulin; produce GLP-1, such as constitutive and/or nutrient-responsive GLP-1 and/or PYY; and/or reduce GIP.

In some embodiments, the patient has non-alcoholic fatty liver disease (NAFLD) and/or non-alcoholic steatohepatitis (NASH), and a treatment of the present inventive concepts can be performed to modify mucosa (e.g. duodenal mucosa) in order to achieve one or more of the following effects: produce insulin, such as a nutrient responsive production of insulin; produce GLP-1, such as constitutive and/or nutrient-responsive GLP-1 and/or PYY; and/or reduce GIP.

In some embodiments, the patient is obese and/or has an obesity related disorder, and a treatment of the present inventive concepts can be performed to modify mucosa (e.g. duodenal mucosa) in order to achieve one or more of the following effects: produce leptin and amylin; produce leptin and PYY, and/or produce leptin and GLP-1, such as constitutive and/or nutrient-responsive GLP-1.

In some embodiments, the patient has polycystic ovarian syndrome (PCOS), and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has hypertriglyceridemia, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has hypercholesterolemia, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has psoriasis, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has coronary artery disease, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has had a stroke, and a treatment of the present inventive concepts can be performed to modify mucosa (e.g. duodenal mucosa) in order to achieve one or more of the following effects: produce insulin, such as a nutrient responsive production of insulin; produce GLP-land/or PYY; and/or reduce GIP.

In some embodiments, the patient has had a transient ischemic attack, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has cognitive decline, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has dementia, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has Alzheimer's Disease, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has neuropathy, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has diabetic nephropathy, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has retinopathy, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has heart disease, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has diabetic heart disease, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has heart failure, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has diabetic heart failure, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has hirsutism, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has hyperandrogenism, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has fertility issues, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has menstrual dysfunction, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has cancer, such as: liver cancer, ovarian cancer, breast cancer, endometrial cancer, cholangiocarcinoma, adenocarcinoma, glandular tissue tumor, stomach cancer, colorectal cancer, and/or prostate cancer, and a treatment of the present inventive concepts can be performed to modify mucosa (e.g. duodenal mucosa) in order to achieve one or more of the following effects: produce GLP-1; reduce GIP; both produce GLP-1 and reduce GIP; produce leptin and amylin; produce leptin and PYY; produce leptin and GLP-1; and/or produce an anti-cancer gene.

In some embodiments, the patient has diastolic dysfunction, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has hypertension, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has had a myocardial infarction, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has microvascular disease related to diabetes, and a treatment of the present inventive concepts can be performed to modify mucosa (e.g. duodenal mucosa) in order to achieve one or more of the following effects: produce insulin, such as a nutrient responsive production of insulin; produce GLP-1 and/or PYY; and/or reduce GIP.

In some embodiments, the patient has anorexia and/or anorexia nervosa, and a treatment of the present inventive concepts can be performed to modify mucosa (e.g. duodenal mucosa) in order to achieve the following effect: reduce GLP-1.

In some embodiments, the patient has a binge eating disorder, and a treatment of the present inventive concepts can be performed to modify mucosa (e.g. duodenal mucosa) in order to achieve following effect: produce GLP-1.

In some embodiments, the patient has hyperphagia or is in a hyperphagic state, and a treatment of the present inventive concepts can be performed to modify mucosa (e.g. duodenal mucosa) in order to achieve the following effect: produce GLP-1.

In some embodiments, the patient has polyphagia, and a treatment of the present inventive concepts can be performed to modify mucosa (e.g. duodenal mucosa) in order to achieve the following effect: produce GLP-1.

In some embodiments, the patient has Prader Willi syndrome and/or an obesity-related genetic disorder, and a treatment of the present inventive concepts can be performed to modify mucosa (e.g. duodenal mucosa) in order to achieve one or more of the following effects: produce leptin; and/or produce ghrelin.

In some embodiments, the patient has hypoglycemia such as post-bariatric hypoglycemia, and a treatment of the present inventive concepts can be performed to modify mucosa (e.g. duodenal mucosa) in order to achieve one or more of the following effects: produce GLP-1, such as constitutive or glucose-responsive GLP-1; and/or produce GIP.

In some embodiments, the patient has recurrent obesity post-bariatric surgery, and a treatment of the present inventive concepts can be performed to modify mucosa (e.g. duodenal mucosa) in order to achieve one or more of the following effects: produce leptin and amylin; produce leptin and PYY, and/or produce leptin and GLP-1.

In some embodiments, the patient has recurrent metabolic disease post-bariatric surgery, and a treatment of the present inventive concepts can be performed to modify mucosa (e.g. duodenal mucosa) in order to achieve one or more of the following effects: produce leptin and amylin; produce leptin and PYY, and/or produce leptin and GLP-1.

In some embodiments, the patient has an iron overload condition such as hemochromatosis types 1-4 and/or bantu siderosis, and a treatment of the present inventive concepts can be performed to modify mucosa (e.g. duodenal mucosa) in order to achieve one or more of the following effects: cause constitutive expression of the HFE gene; disrupt the DMT-1 gene; and/or disrupt a Ferroportin gene.

In some embodiments, the patient has pancreatic cancer, and a treatment of the present inventive concepts can be performed to modify mucosa (e.g. duodenal mucosa) in order to achieve one or more of the following effects: produce GLP-1; reduce GIP; both produce GLP-1 and reduce GIP; and/or produce an anti-cancer gene.

In some embodiments, the patient has short bowel syndrome, and a treatment of the present inventive concepts can be performed to modify mucosa (e.g. duodenal mucosa) in order to achieve one or more of the following effects: improved intestinal function via the introduction of neo-mucosa; and/or produce GLP-2.

In some embodiments, the patient has sleep apnea, and a treatment of the present inventive concepts can be performed to modify mucosa (e.g. duodenal mucosa) in order to achieve one or more of the following effects: produce leptin and amylin; produce leptin and PYY; and/or produce leptin and GLP-1, such as constitutive or glucose-responsive GLP-1.

In some embodiments, the patient has arthritis, and a treatment of the present inventive concepts can be performed to modify mucosa (e.g. duodenal mucosa) in order to achieve one or more of the following effects: produce leptin and amylin; produce leptin and PYY; and/or produce leptin and GLP-1, such as constitutive or glucose-responsive GLP-1.

In some embodiments, the patient has rheumatoid arthritis, and a treatment of the present inventive concepts can be performed to modify mucosa (e.g. duodenal mucosa) in order to achieve one or more of the following effects: produce leptin and amylin; produce leptin and PYY; and/or produce leptin and GLP-1, such as constitutive or glucose-responsive GLP-1.

In some embodiments, the patient has general lipodystrophy (e.g. congenital such as Berardinelli-Seip syndrome or acquired such as Lawrence syndrome), and a treatment of the present inventive concepts can be performed to modify mucosa (e.g. duodenal mucosa) in order to achieve the following effect: produce leptin.

In some embodiments, the patient has familial or acquired partial lipodystrophy (e.g. Barraquer-Simons syndrome or Köbberling-Dunnigan syndrome), and a treatment of the present inventive concepts can be performed to modify mucosa (e.g. duodenal mucosa) in order to achieve the following effect: produce leptin.

In some embodiments, the patient has lipoprotein lipase deficiency (e.g. familial chylomicronemia syndrome, chylomicronemia, chylomicronemia syndrome, and/or hyperlipoproteinemia type Ia), and a treatment of the present inventive concepts can be performed to modify mucosa (e.g. duodenal mucosa) in order to achieve the following effect: produce lipoprotein lipase, such as a constitutive expression of lipoprotein lipase.

In some embodiments, the patient has Hemophilia A, and a treatment of the present inventive concepts can be performed to modify mucosa (e.g. duodenal mucosa) in order to achieve the following effect: produce factor VIII.

In some embodiments, the patient has Hemophilia B, and a treatment of the present inventive concepts can be performed to modify mucosa (e.g. duodenal mucosa) in order to achieve the following effect: produce factor IX.

In some embodiments, the patient has Gaucher's disease, and a treatment of the present inventive concepts can be performed to modify mucosa (e.g. duodenal mucosa) in order to achieve the following effect: produce beta-glucosidase, such as a constitutive expression of beta-glucosidase.

In some embodiments, the patient has Fabry's disease, and a treatment of the present inventive concepts can be performed to modify mucosa (e.g. duodenal mucosa) in order to achieve the following effect: produce alpha-galactosidase A, such as a constitutive expression of alpha-galactosidase A.

In some embodiments, the patient has alpha-1 antitrypsin deficiency, and a treatment of the present inventive concepts can be performed to modify mucosa (e.g. duodenal mucosa) in order to achieve the following effect: produce alpha-1 antitrypsin, such as a constitutive expression of alpha-1 antitrypsin. In some embodiments, a circulating concentration of at least an 11 μM or at least 60 mg per kilogram of patient body weight is achieved.

In some embodiments, the patient has galactosemia type 1, and a treatment of the present inventive concepts can be performed to modify mucosa (e.g. duodenal mucosa) in order to achieve the following effect: produce galactose-a phosphate uridyl transferase, such as a constitutive expression of galactose-a phosphate uridyl transferase.

In some embodiments, the patient has galactosemia type 2, and a treatment of the present inventive concepts can be performed to modify mucosa (e.g. duodenal mucosa) in order to achieve the following effect: produce galactokinase, such as a constitutive expression of galactokinase.

In some embodiments, the patient has galactosemia type 3, and a treatment of the present inventive concepts can be performed to modify mucosa (e.g. duodenal mucosa) in order to achieve the following effect: produce UDP-galactose-4′-epimerase, such as a constitutive expression of UDP-galactose-4′-epimerase.

In some embodiments, the patient has Menkes disease, and a treatment of the present inventive concepts can be performed to modify mucosa (e.g. duodenal mucosa) in order to achieve the following effect: produce ATP7A, such as a constitutive expression of ATP7A.

In some embodiments, the patient has Wilson's disease, and a treatment of the present inventive concepts can be performed to modify mucosa (e.g. duodenal mucosa) in order to achieve the following effect: produce ATP7B, such as a constitutive expression of ATP7B.

In some embodiments, the patient has microvillus inclusion disease, and a treatment of the present inventive concepts can be performed to modify mucosa (e.g. duodenal mucosa) in order to achieve the following effect: produce MYOSB, such as a constitutive expression of MYOSB.

In some embodiments, the patient has congenital tufting enteropathy, and a treatment of the present inventive concepts can be performed to modify mucosa (e.g. duodenal mucosa) in order to achieve one or more of the following effects: produce EpCAM, such as a constitutive expression of EpCAM; and/or produce SPINT2, such as a constitutive expression of SPINT2.

In some embodiments, the patient has chronic gastroparesis, and a treatment of the present inventive concepts can be performed to modify mucosa (e.g. duodenal mucosa) in order to achieve the following effect: produce motilin, such as a constitutive expression of motilin.

In some embodiments, the patient has eosinophilic intestinal disease, and a treatment of the present inventive concepts can be performed to modify mucosa (e.g. duodenal mucosa) in order to achieve the following effect: produce an anti-inflammatory agent, such as a luminal secretion of an anti-inflammatory agent.

In some embodiments, the patient has cystic fibrosis, and a treatment of the present inventive concepts can be performed to modify mucosa (e.g. duodenal mucosa) in order to achieve the following effect: produce CFTR, such as a constitutive expression of CFTR.

In some embodiments, the patient has Crohn's disease, and a treatment of the present inventive concepts can be performed to modify mucosa (e.g. duodenal mucosa) in order to achieve one or more of the following effects: improved barrier function through the introduction of new epithelial cells; and/or reduced inflammatory or immune response to the tissue via the production or reduction of genes involves in inflammation and immune response.

In some embodiments, the patient has inflammatory bowel disease (IBD), and a treatment of the present inventive concepts can be performed to modify mucosa (e.g. duodenal mucosa) in order to achieve one or more the following effects: improved barrier function through the introduction of new epithelial cells; and/or reduced inflammatory or immune response to the tissue via the production or reduction of genes involves in inflammation and immune response. In some embodiments, the patient has eosinophilic esophagitis, and a treatment of the present inventive concepts can be performed to modify mucosa (e.g. duodenal mucosa) in order to achieve the following effect: produce an anti-inflammatory agent, such as a luminal secretion of an anti-inflammatory agent.

In some embodiments, the patient has Celiac Disease, and a treatment of the present inventive concepts can be performed to modify mucosa (e.g. duodenal mucosa) in order to achieve one or more of the following effects: disrupt transglutaminase 2 (TG2) gene; disrupt CCL25 gene; disrupt CXCR3 gene; disrupt MLCK gene; disrupt Claudin-2 gene; produce DPP4, such as a constitutive expression of DPP4; disrupt HLA-DQ2 gene; and/or disrupt RFXANK, RFX5, RFXAP, CIITA, and/or CD74 genes.

In some embodiments, the patient has a medical condition that is caused by deficiencies in proteins that are produced in adipose tissue (which delivers the proteins to the portal circulation), and a treatment of the present inventive concepts can be performed to modify mucosa (e.g. duodenal mucosa) in order to achieve the following effect: produce these otherwise deficient proteins in the intestinal mucosa (e.g. to treat the medical condition).

Disorders of the intestine, such as celiac disease and inflammatory bowel disease, have significant morbidity and mortality for patients around the world. Intestinal procedures in these patients are currently limited to diagnostic procedures, such as mucosal biopsy, or palliative interventional procedures. System 10 can be configured to treat various disorders of the intestine, such as celiac disease and/or inflammatory bowel disease. However, findings by applicant into the role of the mucosa in these diseases, coupled with processes for gene therapy and cell therapy, enable system 10 to provide novel therapies for patients with these diseases.

Material 60 can include cell or other material as described herein, that is mixed with a fluid, such as saline or other biocompatible fluid. In some embodiments, material 60 is mixed with a fluid comprising a volume of at least 1 μL, such as at least 54, 104, 154 and/or 204. In some embodiments, material 60 is mixed with a fluid that is visualizable, as described herein.

In some embodiments, material 60 includes cellular material that can engraft to the deposit site and generate resultant tissue, such as when the deposited material 60 divides, such as through symmetric and/or asymmetric cell division. Progenitor cells in material 60 (e.g. stem cells that can be selected through an enrichment process, such as an enrichment process performed on previously harvested tissue 61) can be used to repopulate and/or reconstitute a neo-mucosa, such as a neo-mucosa with differentiating cells that form a mucosal epithelium at the deposit site and proximate locations (“deposit site” herein). Symmetric division of progenitor cells expand laterally along the surface of the deposit site to form a contiguous epithelial layer. In these embodiments, the resultant tissue (e.g. neo-mucosa) can provide one or more barrier functions, absorptive functions, and/or secretory functions, such as to provide an endocrine function and/or a neuro-endocrine function. These one or more functions can be different than those of the tissue previously present at and/or proximate the deposit site. The absorptive cells in the resultant tissue can enable the absorption of nutrients into the body, such as glucose, amino acids, cholesterol, and the like, for example absorption of a new nutrient and/or a modified (increased or decreased) absorption of a nutrient (such as iron). The secretory function can enable hormonal signaling from this portion of the patient's anatomy (e.g. the patient's intestine) to other body locations, for example a new hormonal signaling and/or modified (increased or decreased) hormonal signaling. For example, the hormonal signaling can modify and/or modulate pancreatic endocrine function (such as the production of insulin and glucagon), pancreatic exocrine function (such as the production of pancreatic digestive enzymes), and the body's insulin resistance, particularly liver insulin resistance. The barrier function provided by the resultant tissue can comprise a barrier function that is different than the barrier function performed by the tissue (e.g. mucosal tissue) of the deposit site prior to the procedure, such as a barrier function that provides different passage of nutrients and/or gut microbiota.

The intestinal lining serves as a barrier function to prevent infectious agents from being transported from the lumen of the gastrointestinal tract into the body. It also serves as an absorptive layer to permit the passage of nutrients, minerals, peptides, fuel (sugars, protein, fat), and bile acids (e.g. in particular portions of the GI tract). The intestinal lining also serves as a signaling organ that communicates signals from the gastrointestinal surface to the rest of the body through neuronal and/or hormonal signals. Different segments of the intestinal lining exhibit different properties in their capacity to absorb different materials as well as in their signaling properties in the fasting and fed states. The harvest site and the deposit site can have similar function in their capacity to serve as a barrier to infection, but may or may not exhibit differences in their absorptive or signaling properties between them.

System 10 can be used to cause resultant tissue (proximate the deposit site) to exhibit one or more properties of the tissue of the harvest site, as described immediately hereabove. The resultant tissue properties can provide a therapeutic benefit to the patient, as described herein. Alternatively or additionally, tissue can be harvested (from any location including the deposit site location), modified ex-vivo (e.g. using processing device 500), such that the resultant tissue proximate the deposit site exhibits one or more properties based on the harvest site and/or the ex-vivo modification. Similarly, this resultant tissue can have properties that provide a therapeutic benefit to the patient.

Material 60 can be deposited at one or more deposit sites to modify the secretions delivered at and/or proximate that deposit site. For example, material 60 can include tissue 61 that is harvested from the terminal ileum, colon, and/or other location, that results, after deposit at a deposit site such as the duodenum and/or proximal jejunum, in increased secretions of peptides that engender insulin sensitization, insulin resistance, and/or satiety (where increased secretions shall include an increase from no secretion of that peptide prior to depositing of material 60). In some embodiments, material 60 can be configured to start and/or increase the secretion of (herein “increase the secretion of”) one or more of: GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; and/or a peptide that engenders insulin sensitization, insulin resistance, and/or satiety. The secretion of these hormones and/or peptides from the resultant tissue to the patient's body can be timed relative to fasting and/or fed states of the patient, such as to improve treatment of the disease. In this way, the resulting hormonal changes in the patient's body are different than before material 60 was deposited (e.g. differences result in the quantity and/or the timing of the secretions).

In some embodiments, depositing of material 60 is configured to stop or at least reduce (herein “reduce”) the secretion of one or more of: GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; a peptide that engenders insulin sensitization, insulin resistance, and/or satiety; and combinations of one, two, or more of these.

Material 60 can be deposited at one or more deposit sites to modify the absorptions that occur at and/or proximate the deposit site, such as to modify the absorption of one or more of: nutrients; fat; protein; glucose; fructose; iron; cholesterol; minerals; peptides; bacteria; virus; fungus; bile salts; bile acids; enzymes (e.g. digestive enzymes produced by the pancreas); micronutrients; macronutrients; pharmacologic agents; alcohol; water; fluids; salt; and/or electrolyte solutions. Treatment could modify absorption for the treated area as well as distal locations in intestine (e.g. modify jejunal and/or ileal absorption by modifying absorption in duodenum).

For example, diarrhea can be treated, such as by increasing absorption of water, electrolyte solutions, and/or other fluids in the small intestine and/or large intestine.

For example, iron absorption could be modified (e.g. reduced), such as to treat an iron overload disorder. In some embodiments, depositing of material 60 is configured to reduce the absorption of one or more nutrients, such as iron.

For example, fat and/or cholesterol absorption could be modified (e.g. reduced), such as a modification in the absorptions that subsequently occur in the distal small intestine, such as to treat a lipid disorder (e.g. hypercholesterolemia).

For example, vitamin B12 absorption could be modified (e.g. increased), such as a modification in the B12 absorptions that occur in patients with pernicious anemia.

For example, chronic gastrointestinal wounds could be treated (e.g. healed), such as by introducing a new population of stem cells to populate the wounded area.

Material 60 can be deposited at one or more deposit sites to modify the neuronal signaling that occurs at and/or proximate the deposit site. For example, an exenatide and/or GLP-1 secretion increase can be generated to induce satiety, delay gastric emptying, increase pancreatic beta cell mass, and/or otherwise cause other physiological changes (e.g. at least partially through neuronal mechanisms). For example, an alteration of signaling to the endocrine pancreas can be generated, such as to treat diabetes and/or improve pancreatic beta cell function in patients with impaired beta cell response to glucose. For example, an alteration of autonomic signaling to the liver and/or adipocyte cells can be generated, such as to treat fatty liver disease and/or obesity or lipodystrophy, respectively. For example, an alteration of autonomic signaling to the vasculature can be generated, such as to treat hypertension, heart failure, and/or diastolic dysfunction. Hormonal signaling may also be altered elsewhere in the gastrointestinal tract, locations remote from the deposit site, through neurohormonal signaling, such as by inducing GLP-1 hormone production in the large intestine and/or the distal small intestine, after depositing material 60 comprising intestinal stem cells at one or more deposit sites in the proximal small intestine.

In some embodiments, depositing of material 60 is configured to modify immune reaction at locations proximate and/or remote from the deposit site. For example, depositing of material 60 can be configured to produce and/or secrete a substance (e.g. an antibody, RNA aptamer, and/or an enzyme) that binds to an antigen known to trigger an autoimmune response in the patient, (e.g. such as an antigen that triggers gluten sensitivity and/or a gluten allergy) such that the depositing of material 60 serves to prevent the patient from experiencing an immune reaction, such as to prevent the patient experiencing a gluten allergy or sensitivity (e.g. for a patient with Celiac disease).

Depositing device 600, harvesting device 400, and/or treatment device 700 (singly or multiply, “devices 600, 400, 700”) can each be configured to be introduced into the body via a natural orifice (e.g. via the mouth or rectum), or via a skin incision (e.g. in an open surgical procedure or a minimally invasive surgical procedure).

Devices 600, 400, 700, can comprise a catheter configuration, such as a catheter that includes an elongate flexible shaft. The shaft can comprise an insertable length configured to access the duodenum, the jejunum, and/or the ileum, such as when placed via the patient's mouth, such as a length of at least 100 cm, 130 cm, or 150 cm, respectively. In some embodiments, harvesting device 400 comprises a longer length than depositing device 600, such as when harvesting device 400 accesses the ileum (e.g. via the mouth) and depositing device 600 accesses the duodenum and/or jejunum (e.g. via the mouth). In some embodiments, harvesting device 400 comprises a shorter length than depositing device 600, such as when harvesting device 400 accesses the colon (e.g. via the rectum) and depositing device 600 accesses the duodenum and/or jejunum (e.g. via the mouth). In some embodiments, harvesting device 400 comprises a shorter length than depositing device 600, such as when harvesting device 400 accesses the duodenum (e.g. via the mouth) and depositing device 600 accesses the jejunum (e.g. via the mouth).

Devices 600, 400, 700 can be configured to be introduced into the patient: through an endoscope (e.g. through a working channel of an endoscope); alongside an endoscope (e.g. through a scope-attached sheath and/or over a guidewire); through a laparoscopic port; and/or via another body access device, such as access device 50 described herebelow.

Depositing device 600 can comprise one, two, or more devices configured to deposit material 60 at a deposit site of a patient. Depositing device 600 can comprise one or more elements for depositing material 60, depositing element 650 shown. Depositing device 600 can include two or more devices that are similar and/or dissimilar (e.g. when a first depositing device 600 and a second depositing device 600 comprise dissimilar lengths, and/or dissimilar depositing elements 650). The material deposited by depositing device 600, material 60 shown in FIG. 1 and described herebelow, can include tissue 61, processed tissue (e.g. tissue 61 processed as described herein), an agent (e.g. a pharmaceutical agent or other agent 62 as described herein), and/or other material (e.g. a material configured to provide and/or support a diagnostic or therapeutic benefit).

Material 60 can be deposited at one, two, or more deposit sites of a patient. Deposit sites can include, but are not limited to: luminal wall tissue of gastrointestinal (GI) tract; mucosal tissue of GI tract; submucosal tissue of GI tract; and/or the peritoneal cavity. Deposit sites can include but are not limited to: the gastro intestinal tract; the mouth; the esophagus; the stomach; the duodenum; the jejunum; the ileum; the colon; an organ; the brain; the lungs; the liver; the bladder; the kidneys; the heart; the intestines; the skin; and/or the peritoneal cavity.

In some embodiments, depositing element 650 comprises at least two depositing elements 650a,b, and/or at least three depositing elements 650 a,b,c. The multiple depositing elements 650 can be configured to deposit material 60 simultaneously and/or sequentially. Depositing element 650 can comprise one, two, three, or more needles through which material 60 can be deposited at one or more deposit sites. Alternatively or additionally, depositing element 650 can comprise one, two, three, or more fluid jets through which material 60 is deposited at one or more deposit sties. In some embodiments, depositing element 650 comprises one or more material 60 depositing elements 650 positioned on an expandable element, such as an inflatable balloon, a flexible basket or cage, a series of radially deployable arms, and/or an unfurlable sheet. In some embodiments, depositing device 600 is configured to lift tissue (e.g. expansion of submucosal tissue via injection into the submucosa of a balanced salt solution such as normal saline), prior to the depositing of material 60.

In some embodiments, depositing device 600 is configured to maintain (e.g. to protect) material 60 at the deposit location (e.g. prevent the migration of material 60 from the deposit site). For example, depositing device 600 can include a gel configured to be applied on top of the delivered material 60 and/or a sleeve configured to be placed over the material 60 (e.g. a sleeve placed in a lumen whose wall has received material 60).

In some embodiments, material 60 includes a carrier element, carrier 63 described herein, such as an adhesive, clip, stent, tubular structure, and/or other carrier element, and depositing device 600 deploys carrier 63 to deposit material 60.

In some embodiments, depositing device 600 is configured to deposit material 60 along one or more deposit sites with a cumulative length of at least 25 mm. In some embodiments, material 60 is deposited within a cumulative surface area (e.g. surface area of the inner wall of one or more segments of the GI tract) of at least 50 cm², at least 100 cm², or at least 250 cm² (e.g. material is deposited into one or more “patches” that cover 1% to 100% of that surface area).

In some embodiments, depositing device 600 is of similar construction arrangement to a device described in applicant's co-pending application International PCT Patent Application Number PCT/US2018/042438 (Attorney Docket No. 41714-715.601, Client Docket No. MCT-025-PCT), entitled “Intestinal Catheter Device and System”, filed Jul. 17, 2018, the contents of which is incorporated herein by reference in its entirety for all purposes.

Harvesting device 400 can comprise one, two, or more devices configured to harvest tissue 61 at a harvest site of a patient. Harvesting device 400 comprises one or more elements for harvesting tissue 61, harvesting element 450 shown. Harvesting device 400 can include two or more devices that are similar and/or dissimilar (e.g. when a first harvesting device 400 and a second harvesting device 400 comprise dissimilar lengths and/or dissimilar harvesting elements 450). Harvesting device 400 can comprise a device similar to a device used to perform endoscopic mucosal resection (EMR) procedures and/or endoscopic submucosal dissection (ESD) procedures. Harvesting device 400 can be configured to obtain one, two, or more tissue 61 samples. Harvesting device 400 can be configured to perform a core or punch biopsy of tissue (e.g. mucosal tissue). In some embodiments, harvesting device 400 is configured to simultaneously obtain multiple samples of tissue 61 (e.g. multiple simultaneous core and/or punch biopsies). In some embodiments, harvesting device 400 comprises a suction and/or guillotine biopsy device. In some embodiments, harvesting device 400 comprises a device configured to scrape a tissue surface, such as to harvest tissue of the mouth or other body location.

Harvesting device 400 can be configured to obtain individual tissue samples with one or more dimensions selected from the group consisting of: a width of less than 3 mm; a thickness (e.g. tissue depth) of at least 500 microns or at least 600 microns; a thickness of at least 1 mm; a thickness of at least 1.5 mm; and combinations of one, two, or more of these.

Harvesting element 450 can comprise one, two, or more elements configured to capture tissue, such as one or more: needles (e.g. one or more needles to which a vacuum can be applied); biopsy elements; tissue grasping elements; vacuum elements; cutting elements; mucosal lifting elements, and/or dissecting elements. In some embodiments, harvesting element 450 comprises at least two harvesting elements 450a,b, and/or at least three harvesting elements 450a,b,c. The multiple harvesting elements 450 can be configured to harvest tissue simultaneously and/or sequentially.

In some embodiments, harvesting device 400 is of similar construction and arrangement to a device described in applicant's co-pending application International PCT Patent Application Number PCT/US2018/042438 (Attorney Docket No. 41714-715.601, Client Docket No. MCT-025-PCT), entitled “Intestinal Catheter Device and System”, filed Jul. 17, 2018, the contents of which is incorporated herein by reference in its entirety for all purposes.

Tissue 61 can comprise material harvested from one, two, or more anatomical locations of a patient. Anatomical locations for harvest sites include but are not limited to: the gastro intestinal tract, the mouth; the esophagus; the stomach; the duodenum, the jejunum, the ileum, the colon, an organ, the brain, the lungs, the liver, the bladder, the kidneys, the heart, the intestines, the skin, and/or the peritoneal cavity. Tissue 61 can comprise autograft tissue, autogenous tissue, autologous tissue, allograft tissue, and/or xenograft tissue.

Tissue 61 (e.g. tissue 61 captured by harvesting device 400) can comprise: mucosal tissue; submucosal tissue; tissue comprising at least one stem cell; tissue comprising at least one stem cell of the mucosa; tissue comprising at least one mucosal crypt containing a stem cell; mucosal tissue comprising at least one stem-cell containing crypt; organoids; epithelial layer tissue; uroepithelial layer tissue; intestinal epithelial layer tissue; and/or lung epithelial layer tissue.

In some embodiments, harvesting device 400 is configured to harvest tissue 61 (e.g. obtain individual tissue samples) that preferentially contain pluripotent stem cells and/or preferentially do not contain terminally differentiated cells of the intestinal mucosa. In some embodiments, harvesting device 400 is configured to harvest tissue 61 (e.g. obtain individual tissue samples) that also contain elements of the local microbiome of the harvest site. Alternatively, harvesting device 400 can be configured to harvest tissue 61 that preferentially do not contain elements of the local microbiome of the harvesting location, such as by harvesting tissue 61 that has been pre-treated by a component of system 10 (e.g. pre-treated by a component of harvesting device 400) to reduce its resident microbiome population (e.g. a component that disinfects the tissue to be harvested).

Tissue 61 can comprise hormonal activating tissue. Alternatively or additionally, tissue 61 can comprise hormonal deactivating tissue.

In some embodiments, tissue 61 does not include tissue of (e.g. harvesting device 400 avoids harvesting tissue from): the lower esophageal sphincter; the pylorus; the ampulla of Vater; the ileocecal valve; and combinations of one, two, or more of these.

Tissue 61 can comprise captured tissue that has at least 1 cm² of surface area (e.g. at least 1 cm² of mucosal tissue luminal surface area), which can be harvested by harvesting device 400 in a single step or multiple steps. The term “surface area”, when used to describe the surface area of a segment of sampled tissue that has a relatively flat geometry (e.g. a length and width much greater than its thickness), is to be taken as the surface area of one side of the sample. When used to describe the surface area of multiple samples, each with a relatively flat geometry, “surface area” is to be taken as the cumulative surface areas of a single side of each sample.

In some embodiments, tissue 61 comprises a minimum and/or maximum amount of tissue to be harvested, such as an amount selected from the group consisting of: at least one core biopsy; no more than 20 core biopsies; at least 1000 cells; no more than 1 billion cells; and combinations of one, two, or more of these.

Treatment device 700 can comprise one, two, or more devices configured to ablate, remove, modify, expand, and/or otherwise treat tissue at a treatment site of a patient. Treatment device 700 comprises one or more elements for treating tissue of the patient, treatment element 750 shown. Treatment device 700 can include two or more devices that are similar and/or dissimilar (e.g. when a first treatment device 700 and a second treatment device 700 comprise dissimilar lengths and/or dissimilar treatment elements 750). In some embodiments, treatment device 700 comprises a first treatment device 700a that causes tissue to necrose (e.g. via delivery of thermal energy, such as heat energy and/or cryogenic energy; electrical energy; and/or a chemical agent to tissue), and a second treatment device 700b that provides an abrasive force to the necrosed tissue. The second treatment device 700b can be used in the same clinical procedure as first treatment device 700a is used, or in a subsequent clinical procedure (e.g. a second clinical procedure 1, 2, or more days after the first clinical procedure). In these embodiments, tissue can be removed proximate (e.g. at and/or near) one or more deposit sites at which material 60 is to be deposited (e.g. deposited using depositing device 600).

The tissue treated by treatment device 700 can include mucosal tissue; submucosal tissue; tissue comprising at least one stem cell; tissue comprising at least one stem cell of the mucosa; tissue comprising at least one mucosal crypt containing a stem cell; and/or mucosal tissue comprising at least one stem-cell containing crypt. Tissue treated by treatment device 700 can include tissue of one or more anatomical locations selected from the group consisting of: the gastro intestinal tract; the mouth; the esophagus; the stomach; the duodenum; the jejunum; the ileum; the colon; an organ; the brain; the lungs; the liver; the bladder; the kidneys; the heart; the intestines; the skin; the peritoneal cavity; and combinations of one, two, or more of these. Treated tissue by treatment device 700 can comprise tissue that activates and/or deactivates hormonal signals and/or signaling pathways. In some embodiments, tissue treated by treatment device 700 does not include tissue of (e.g. treatment device 700 avoids adversely effecting tissue from): the lower esophageal sphincter; the pylorus; the ampulla of Vater; the ileocecal valve; and combinations of one, two, or more of these.

Treatment device 700 can be used to perform a tissue modification procedure as defined hereabove, such as a tissue modification procedure performed proximate (e.g. at and/or near) one or more intended deposit sites for material 60. In some embodiments, treatment device 700 is configured to ablate and/or otherwise remove tissue from the deposit site (e.g. prior to the depositing of material 60), such that resultant tissue (regrowth of tissue after the treatment performed by treatment device 700) includes tissue properties that are “driven by” the characteristics of material 60. In these embodiments, the removal of tissue can reduce the effects (e.g. competing effects) of the tissue previously present at the deposit site (e.g. tissue removed using treatment device 700).

In some embodiments, a tissue modification procedure is performed using treatment device 700 at a location distal to a deposit site and/or proximal to a deposit site. In these embodiments, a tissue modification procedure may or may not also be performed at the deposit site.

In some embodiments, a tissue modification procedure is performed proximal to the deposit site (e.g. upstream in the GI tract), such as to protect the deposited material (e.g. by reducing the intraluminal contents from overexposing the site, such as for a period of 2 weeks in which food intake is limited). For example, treatment device 700 can be configured to perform a luminal narrowing procedure in which one or more materials are injected into a full or partial circumferential portion of an axial segment of the GI tract, as described herein, such as to restrict the patient's food intake and/or to modify the flow of contents within the lumen of the GI tract. Alternatively or additionally, a tissue modification procedure can be performed distal to the deposit site (e.g. downstream in the GI tract). For example, treatment device 700 can be used to perform a luminal narrowing procedure at an axial segment of the GI tract downstream from the deposit site, such as to cause the deposit site to be washed or bathed by contents passing therethrough (e.g. washing or bathing that results from intraluminal contents remaining at the deposit site for a longer period of time due to the downstream narrowed segment).

Treatment element 750 can comprise one, two, three, or more treatment elements configured to ablate, remove, resurface, denature, and/or otherwise effect tissue, such as mucosal tissue. Treatment element 750 can deliver an ablative fluid to treat the tissue (e.g. an ablative fluid applied directly to the tissue or delivered to a balloon placed in contact with tissue). Treatment element 750 can deliver energy to tissue, such as electrical energy; magnetic energy; chemical energy; sound energy; and/or light energy. In some embodiments, treatment element 750 comprises multiple treatment elements arranged in a circumferential pattern and/or a single element that treats a circumferential segment of tubular tissue (e.g. delivers energy and/or an agent to the full circumferential wall of a segment of intestine). The depositing of material 60 can occur before and/or after the use of treatment element 750, such as by injecting material 60 into the submucosa and subsequently performing a treatment with treatment element 750, and/or by performing a treatment with element 750 and then depositing material 60. Treating and depositing steps can be performed in the same procedure or in different procedures. These steps can be performed within minutes of one another, within 3 days, and/or within 5 days.

In some embodiments, treatment device 700 is of similar construction arrangement to a device described in applicant's co-pending application International PCT Patent Application Number PCT/US2018/042438 (Attorney Docket No. 41714-715.601, Client Docket No. MCT-025-PCT), entitled “Intestinal Catheter Device and System”, filed Jul. 17, 2018, the contents of which is incorporated herein by reference in its entirety for all purposes.

System 10 can further comprise a device configured to provide access within the patient, access device 50. Access device 50 can comprise an endoscope, an endoscope-attached sheath, a laparoscopic port, a vascular introducer, and/or another patient access device. Access device 50 can further comprise one or more guidewires, e.g. one or more guidewires over which devices 600, 400, and/or 700 are introduced into the patient (e.g. and subsequently withdrawn from the patient), such as by using standard “over the wire” clinical techniques. Access device 50 can comprise a camera, such as camera and a display, such as when access device 50 comprises an endoscope.

In some embodiments, material 60 comprises tissue 61 that is not processed. Alternatively or additionally, material 60 can comprise processed tissue 61, such as when tissue 61 is processed by processing device 500. The term “material 60”, as used herein, shall include a partially processed material 60, such as a material that is to be further processed prior to implantation in the patient at the deposit site. The term “material “60”, as used herein, shall include “resultant material”, as defined herein.

Processing device 500 can be configured to extract, isolate, separate, and/or otherwise collect particular cells from tissue 61, such as stem cells.

Processing device 500 can be configured to generate new cells, such as to amplify the number of cells in a sample (e.g. amplify the number of stem cells). Processing device 500 can be configured to generate an amplified volume of tissue 61, such as an amplification of at least 10 fold, at least 100 fold, and/or at least 1,000,000 fold the number of cells harvested. This amplification process can be performed over a duration of least 1 day, 3 days, 15 days, and/or 30 days. Material 60 generated by the amplification process can be deposited in the patient within 1 day, 3 days, 15 days, 30 days, and/or 90 days of the amplification process. Alternatively, material 60 can be stored indefinitely, and eventually deposited in the patient at any point after the amplification process. Processing device 500 (or another component of system 10) can include a freezing component, wherein material 60 is frozen (e.g. and stored at less than 0° C., such as storage at a temperature less than −20° C., less than −80° C., or at a temperature of approximately −200° C.). In some embodiments, material 60 is rapidly frozen (e.g. flash-frozen in less than 5 seconds, such as freezing using liquid nitrogen) such as to optimize viability of a majority of the tissue.

Processing device 500 can be used to collect stem cells (e.g. intestinal stem cells) from tissue 61 comprising the mucosa (e.g. the deep mucosa), submucosa and/or overlying mucosa/villous structure. For example, the collected mucosa can be shaved, and the submucosa mechanically dissected. Stem cells can be grown on and/or in a gel-based matrix, such as to generate a solid and/or semi-aqueous product form.

Processing device 500 can be used to amplify stem cells (e.g. intestinal stem cells) outside of the patient, it and can include components to suspend the result in a saline or balanced salt solution.

Processing device 500 can be configured to perform an ex-vivo enriched cell culturing process on tissue 61, such as a process in which a medium that includes trophic factors is used.

Processing device 500 can be configured to perform an encapsulation, such as to include cells in a scaffold or other cell-carrying component.

Processing device 500 can be configured to perform cell sorting, such as to dissociate cells, suspend them in solution, and use a cell sorting mechanism to select stem cells. Stem cells can be selected based on cell surface receptors, and they then can be amplified prior to depositing at one or more deposit sties.

Processing device 500 can be configured to remove collagen from tissue 61, such as to isolate crypts.

Processing device 500 can be configured to cause (e.g. allow) cells (e.g. cells of tissue 61) to grow into a material 60 comprising organoids and/or it may cause the cells to grow into a material 60 comprising a monolayer sheet of cells. The organoids may be dissociated prior to deposition of material 60 and/or they may be maintained as organoids at the time of material 60 deposition.

Processing device 500 can combine tissue 61 with an agent, such as an agent 62 comprising a material selected from the group consisting of: trophic factor; antioxidant; salicylate; a nonsteroidal anti-inflammatory drug (NSAID); and combinations of one, two, or more of these. In some embodiments, agent 62 is added at a time near the time material 60 is deposited in the patient (e.g. within 8 hours of the depositing of material 60 at the deposit site).

Processing device 500 can include one or more scaffolds, such as a scaffold in which tissue is grown (e.g. in combination with cell culturing). The scaffold may comprise a cellular or acellular scaffold. The scaffold may comprise small intestinal tissue from a human or other mammal. The scaffold can comprise small intestinal submucosa, such as porcine small intestinal submucosa. The scaffold can comprise a gelatinous protein mixture configured as a basement membrane matrix (e.g. Matrigel or Cultrex BME). This basement membrane matrix (also referred to herein as “basement membrane”) can comprise a shear thinning hydrogel.

Processing device 500 can be configured to arrange cells in a hydrogel matrix, such as when material 60 includes the hydrogel matrix (e.g. the hydrogel matrix is deposited at the deposit site). The hydrogel matrix can be configured to degrade over time in the patient.

Processing device 500 can be configured to modify tissue (e.g. tissue 61 and/or other tissue) in one or more ways, such as to genetically, chemically, and/or epigenetically modify tissue 61.

Processing device 500 can be configured to perform a transgenic treatment of tissue (e.g. a transgenic modification of tissue 61 and/or other tissue), to cause a targeted expression (e.g. an increase or decrease in expression) in the tissue generated by depositing material 60 (e.g. a transgenic transformation causes the generated tissue to hyper-secrete and/or hypo-secrete desired hormones). For example, processing device 500 can perform a transgenic treatment to cause the resultant tissue (tissue generated by depositing of material 60) to express exenatide or GLP-1 or a GLP-1 analogue, such as when system 10 is configured to treat obesity, Type 2 diabetes (with or without obesity also being present), non-alcoholic fatty liver disease (NAFLD), non-alcoholic steatohepatitis (NASH), and/or early stage Type 1 diabetes (e.g. in a patient with sufficient residual beta cell mass, such as to maintain and/or increase that mass). In these embodiments, processing device 500 can further cause the deposited material 60 to express PYY and/or oxyntomodulin or Glicentin (to enrich and/or de-enrich the tissue for certain types of enteroendocrine cells, to enhance, suppress, and/or modify the hormonal contents of enteroendocrine secretion), such as when system 10 is configured to treat Type 2 diabetes. In some embodiments, processing device 500 can perform a transgenic treatment to cause the resultant tissue (that is generated based on the depositing of material 60) to express (e.g. start or at least increase the secretion of): GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; and/or a peptide that engenders insulin sensitization, insulin resistance, and/or satiety. In some embodiments, a transgenic treatment of tissue is performed on tissue 61 to cause the resultant tissue to express the above hormones in response to luminal contents (e.g. nutrients, bile salts, and the like), and/or express these hormones in response to blood components (e.g. glucose). The deposited material 60 can be configured to express antibodies that inhibit the action of one, two, or any combination of these proteins. Inhibition of select proteins and activation or expression of other proteins can be performed combinatorially. The combinatorial use can be determined by the patient disease state and/or some other measurable physiologic parameter, such as insulin resistant state, diabetes status, blood sugar levels, liver fat, liver fibrosis, fertility, BMI, and/or other factors.

Processing device 500 can perform a transgenic modification that includes Crispr/Cas9 gene editing of the somatic DNA or similar gene editing tools that perform a similar function to CRISPR/Cas9 gene editing. For example, processing device 500 can perform a transgenic modification that includes use of a piggyBac transposon, lentiviral vector, adenovirus, and/or AAV vector.

Processing device 500 can perform processing that includes decontamination of tissue to alter the microbial content of the tissue (e.g. harvested tissue 61) prior to material 60 being deposited in the patient. This decontamination can be achieved in a variety of ways, such as the incubation of the cells with antibiotics for a specific length of time, such as at least 1 day, 2 days, or 5 days. The process can involve replacing the culture media in which the tissue is growing, such as to a culture media that does not contain antibiotics. Optionally, the processing performed by processing device 500 can involve a confirmatory step to assure that material 60 does not include any infective material, such as a confirmatory step including a test for endotoxins.

Processing device 500 can be configured to eliminate or at least reduce (“reduce” herein) microbes, such as microbes that are included in the harvesting of tissue 61. For example, tissue 61 can be processed in an antibiotic medium, and/or microbes can be strained with a filter (e.g. a filter in which microbes pass through but larger cells, such as stem cells, do not).

Processing device 500 can be configured to perform a “tagging” of tissue, such that material 60 includes a fluorescent or other marker used to identify material 60 (and/or resultant material, also referred to as “material 60” herein) after depositing in the patient. Tagging can be used to assess procedure longevity, to assess the efficacy of the depositing procedure, and/or to identify tissue to be removed in a subsequent procedure if desired (e.g. an undesired effect is encountered and should be reversed or reduced).

Processing device 500 can be configured to add an agent 62 comprising a bioadhesive agent (e.g. a bioadhesive polymer) to material 60, such as an agent configured to cause or enhance adhesion of material 60 to a tissue surface at a deposit site.

In some embodiments, processing device 500 is configured to treat in-situ tissue prior to its harvest. For example, processing device 500 can be inserted into the patient's GI tract, such as to allow an operator to inject one or more agents (e.g. agent 62 and/or 70 described herein) proximate tissue to be harvested (e.g. tissue 61 harvested at least 1 day after the injection).

Material 60 can comprise tissue 61, and/or processed tissue 61 (e.g. tissue 61 that has been modified and/or otherwise processed by processing device 500 as described herein). As used herein, the term “tissue 61” can comprise unprocessed tissue 61 and/or processed tissue 61.

In some embodiments, material 60 can comprise one or more components that are configured to pass through the membrane of cells at a deposit site.

In some embodiments, material 60 does not include tissue 61. For example, material 60 can comprise a transgenic virus and/or other tissue modifying material that is applied to and/or within a deposit site.

In some embodiments, material 60 comprises one or more agents, such as agent 62 described herein.

In some embodiments, material 60 includes one or more carrier elements 63 configured to aid in the depositing of material 60 and/or to maintain material 60 at the deposit site.

For example, carrier 63 can comprise a deployment device, carrier 63a, such as a stent-like device onto which the other materials of material 60 (e.g. tissue 61) is deposited, the stent-like device deployed within a lumen (e.g. the lumen of the small intestine or other body lumen) at the deposit site location. Carrier 63a can comprise a full or partial circumferential tubular structure, such as a structure seeded with tissue 61. Carrier 63a can comprise a tubular structure comprising a hydrogel.

Alternatively or additionally, carrier 63 can comprise an adhesive, adhesive 63b, such as an adhesive gel, such as an adhesive gel used to secure material 60 to the deposit site and/or to secure multiple components of material 60 together.

Alternatively or additionally, carrier 63 can comprise a coating or wrap, coating 63c, such as a coating or wrap configured to prevent material 60 from migrating from the deposit site (e.g. at an undesired time). In some embodiments, carrier 63 comprises a carrier 63a, and a coating 63c configured as a protective coating or other protecting element. In some embodiments, carrier 63 comprises a coating 63c that is applied to a deposit site in one or more procedures performed after the depositing of material 60 at the deposit site (depositing of a material 60 with or without coating 63c). In these embodiments, carrier 63a protects other components of material 60 (e.g. tissue 61), such as to prevent undesired migration of material 60.

Material 60 can comprise tissue provided en bloc.

Material 60 can comprise biobanked tissue. For example, material 60 can comprise tissue that is treated, and then stored (e.g. frozen), and/or tissue that is stored (e.g. frozen), and subsequently treated.

Agent 62 can comprise one or more pharmaceutical drugs, nutrients (e.g. hexoses, lipids, and/or amino acids), vitamins (e.g. water-soluble vitamins such as ascorbic acid), buffering agents, chemicals, fillers, and/or other agents that are included in material 60. In some embodiments, agent 62 comprises one or more agents selected from the group consisting of: antibiotic; adhesive agent such as a bio-adhesive agent; a trophic agent (e.g. a growth factor or other factor used to promote wound healing); a shielding agent (e.g. an agent configured to protect one or more components of material 60 after depositing); and combinations of one, two, or more of these.

In some embodiments, one or more agents 62 are included in a process for creating material 60 at a time that is proximate the time that material 60 is deposited at a deposit site, such as a time within 8 hours of the depositing of material 60 at a deposit site.

In some embodiments, system 10 includes one or more pharmaceutical drugs or other agents, agent 70 that is delivered to the patient prior to the depositing of material 60, and/or after the depositing of material 60. Agent 70 can be delivered to the patient orally, transdermally, via an injection (e.g. a subcutaneous, intramuscular, epidural, and/or intrathecal injection); and/or intravascularly (e.g. intravenously and/or intraarterially).

In some embodiments, agent 70 comprises one or more of: antibiotics; probiotics; and/or prebiotics.

In some embodiments, agent 70 comprises iron, such as when material 60 comprises tissue 61 that has been harvested by harvesting device 400 from the ileum, and depositing device 600 deposits material 60 in the duodenum and/or proximal jejunum. In these embodiments, the iron-based agent 70 can be delivered to the patient to prevent an iron insufficiency otherwise caused by the depositing of material 60.

In some embodiments, agent 70 comprises one or more of: an anti-inflammatory agent; an NSAID, and/or an immunosuppressant.

In some embodiments, depositing device 600 comprises one or more functional elements 699. In some embodiments, system 10 comprises a harvesting device 400 that comprises one or more functional elements 499. In some embodiments, system 10 comprises a processing device 500 that comprises one or more functional elements 599. In some embodiments, system 10 comprises a treatment device 700 that comprises one or more functional elements 799. Functional elements 499, 599, 699, and/or 799 can comprise one, two, or more sensors, transducers, and/or other functional elements as described herein.

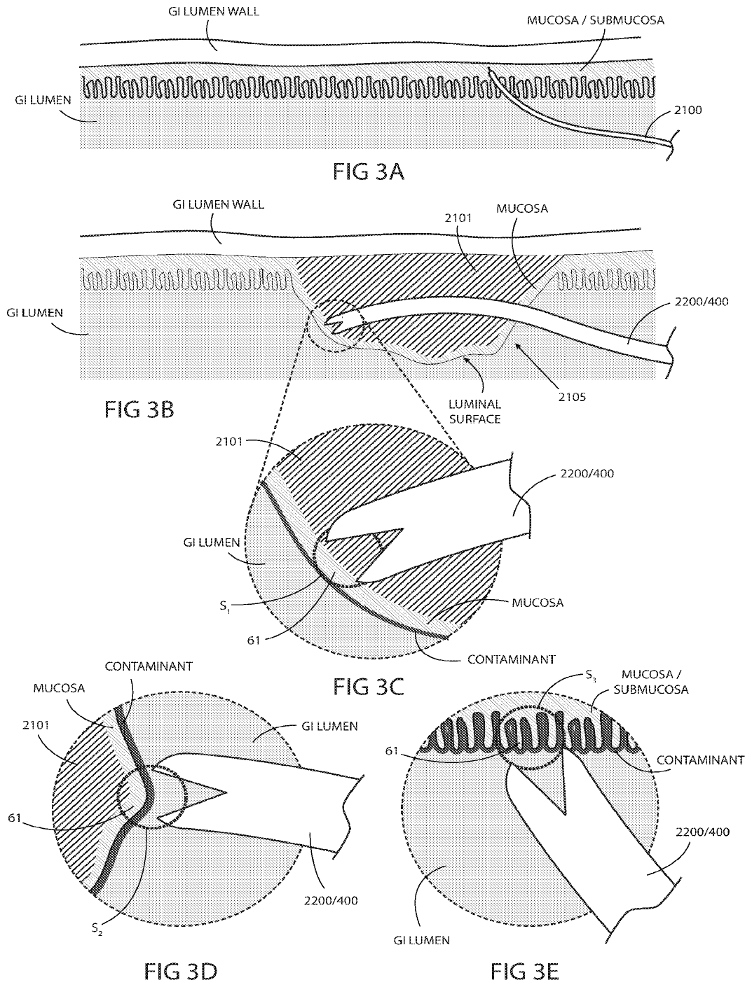

In some embodiments, the depositing of material 60 at a deposit site includes performing a tissue expansion procedure proximate the deposit site, such as a tissue expansion procedure in which a fluid such as saline is introduced into the tissue prior to, during, and/or after depositing of material 60 at the deposit site. This tissue expansion procedure can be performed to enhance the distribution of material 60 subsequently introduced to the deposit site. This tissue expansion procedure can be performed using a device (e.g. treatment device 700 and/or depositing device 600 described in reference to FIG. 1, and/or expansion device 2100 described herebelow in reference to FIGS. 3A-E) that includes one, two, three or more fluid delivery elements (e.g. needles and/or waterjets). In some embodiments, the fluid delivery elements that deliver the expansion fluid also deliver material 60. In some embodiments, tissue expansion is performed at a first axial segment of the GI tract, and then performed at one or more additional axial segments of the GI tract (e.g. via advancement and/or retraction of the tissue expansion device). Alternatively or additionally, tissue expansion can be performed at a first axial location, the tissue expansion device can be rotated, and tissue expansion can be performed again at that axial location, at a different circumferential portion. In each of these embodiments, material 60 can be deposited immediately after each tissue expansion (e.g. expand at a first site then deposit at the first site, subsequently expand at a second site and then deposit at that second site, and so on, for example after rotation and/or translation of depositing device 600 after each deposit). Alternatively or additionally, two or more expansions of axial and/or circumferential segments can be performed, after which material 60 is deposited at those two or more expansion locations. The amount of fluid delivered proximate each deposit site can comprise a volume of at least 1 ml, at least 5 ml, at least 10 ml, at least 15 ml, or at least 20 ml. In some embodiments, fluid is delivered by multiple (e.g. 2 or 3) fluid delivery elements (e.g. delivered simultaneously to cover a full circumferential portion of an axial segment of the GI tract), such as when each fluid delivery element (e.g. two or more of elements 450a,b,c and/or 650a,b,c) delivers a volume of at least 1 ml, at least 5 ml, at least 10 ml, at least 15 ml, or at least 20 ml. The fluid delivered can include a visualizable agent, such as an agent visualizable by a visible light camera (e.g. methylene blue), a fluorescent agent, an agent visualizable by an infrared camera, an agent visualizable by ultrasound, and/or an agent radiographically visualizable. In some embodiments, alternating steps of expanding tissue (e.g. submucosal tissue) and delivering material 60 are performed.

In some embodiments, the tissue treatment performed proximate a deposit site and the depositing of material 60 at a deposit site are performed with the same device, such as when depositing device 600 and treatment device 700 comprise the same device. In some embodiments, material 60 is present (e.g. previously loaded) in the combined device (“device 600/700” hereinafter) when a tissue treatment is being performed. For example, material 60 can be loaded into a lumen of the device 600/700 that also includes injectate (e.g. injectate 2101 described herein) to be delivered proximate the deposit site, the injectate to be delivered (e.g. in a tissue expansion procedure) prior to delivery of material 60. In some embodiments, the tissue treatment procedure comprises a procedure at a temperature quite different than body temperature (e.g. to support a heat ablation procedure or a cryogenic ablation procedure). In some embodiments, a component of material 60 (e.g. a basement membrane matrix of material 60) could be configured to at least partially solidify (e.g. when heated), the solidification providing a protection to cells of material 60. The solidification can be configured to improve delivery of material 60 to the deposit site (e.g. improve passage of material 60 through one or more needles or other fluid delivery elements such as elements 450a,b,c and/or 650a,b,c described herein), such as to avoid damage (e.g. cellular death) of one or more components of material 60. In some embodiments, heat provided by device 600/700 is used to promote cellular growth of material 60. Heat provided to material 60 by device 600/700 can comprise a temperature in the range of at least 30° C., at least 40° C., and/or at least 60° C. Alternatively, in some embodiments, material 60 is protected from undesired hot (or cold) temperatures encountered when device 600/700 treats tissue, such as when material 60 is introduced (loaded) into device 600/700 after a thermal tissue ablation is performed. In some embodiments, device 600/700 is maintained in the same axial position in the GI tract during the tissue treatment and material 60 deposition steps, such as via vacuum ports included in device 600/700 (e.g. vacuum ports into which tissue is captured during a tissue expansion procedure).

In some embodiments, system 10 includes diagnostic kit 81 which includes one or more components configured to perform an analysis, such as an analysis of patients P1 and/or P2 (e.g. tissue 61 of patient P2), and/or an analysis of material 60. Diagnostic kit 81 can include components (e.g. equipment and/or supplies) configured to perform multiple tests during the creation of material 60 and/or the depositing of material 60 into patient P1, such as is described herebelow in reference to FIG. 2.

In some embodiments, diagnostic kit 81 comprises components used to perform a screening endoscopy (e.g. a screening endoscopy performed in STEPs 1010 and 1220 described herebelow in reference to FIG. 2), such as when diagnostic kit 81 comprises an endoscope. The endoscopy can be used to screen out patients with cancer, an infection, and/or other gastrointestinal pathology. The endoscopy can be used to identify a harvest site and/or a deposit site, and/or to confirm tissue 61 has been harvested from a desired anatomical location and/or material 60 has been adequately deposited in a desired anatomical location.

Diagnostic kit 81 can comprise one or more components configured to perform a “tissue test”, such as a test to determine whether desired and/or undesired conditions of the tissue are present. In some embodiments, a sample of tissue is tested to confirm the absence of: infected tissue; undesired bacteria; endotoxins and/or other toxins; cancerous tissue; mycoplasma; undesired proteins (e.g. GIP, GLP-1, and/or other incretins, such as a test including a response to a glucose-based stimulus); a virus; undesired bacteria; E. coli; an adventitious agent; and/or other undesirable tissue characteristic. For example, this testing can be used to screen for a medical condition (e.g. disease and/or disorder) selected from the group consisting of: cancer (e.g. cancer of the GI tract); infection (e.g. an infection of the GI tract); presence of Clostridium difficile bacteria (C. difficile); HIV; Hepatitis virus A, B, and/or C; syphilis; tuberculosis; and combinations of one, two, or more of these. Alternatively or additionally, a tissue test performed using diagnostic kit 81 can comprise a test of tissue to confirm desired characteristics of the tissue are present, such as to confirm tissue is from a particular donor (e.g. patient P2 of the present inventive concepts), and/or to confirm the presence of desired material, such as a material selected from the group consisting of: desired proteins (e.g. GIP, GLP-1, and/or other incretins, such as a test including a response to a glucose-based stimulus); immune cells; stem cells; enteroendocrine cells; a genetic sequence; an mRNA expression; cell surface antigens; and combinations of these. A tissue test comprising a donor confirmation test performed using diagnostic kit 81 can comprise a test selected from the group consisting of: DNA test; mRNA assay; proteomics assay; flow cytometry assay; immunohistochemical analysis; enzyme-linked immunosorbent assay (ELISA); and combinations thereof. In some embodiments, diagnostic kit 81 comprises components configured to perform a test for mycoplasma and/or or virus, and the kit 81 is used to test a culture in which material 60 is expanded (e.g. in STEP 1060 and/or STEP 1080 described herebelow in reference to FIG. 2).

Diagnostic kit 81 can include components configured to assess a parameter related to an expansion of tissue, (e.g. as performed in STEPs 1060 and/or 1080 described herebelow in reference to FIG. 2), components configured to assess a quantity of tissue (e.g. a quantity of cells present in a sample), and/or components configured to assess a concentration and/or ratio of one or more substances of tissue. For example, during and/or after material 60 expansion, diagnostic kit 81 can be configured to assess a parameter (e.g. to confirm adequate quantity and/or confirm other desired expansion parameter) selected from the group consisting of: cell growth rate; organoid growth rate; organoid density (e.g. in growth substrate); morphometry of organoids, including a quantification of the number of buds and/or crypts in the organoids; cell culture media secretions (e.g. GIP, GLP-1, insulin and/or other marker peptide not normally secreted by this cell type); and combinations of these. Diagnostic kit 81 can include components to assess a tissue parameter selected from the group consisting of: number of crypts in a tissue sample; basement membrane matrix seeding density (e.g. derived from crypt count); presence and/or concentration of immune cells; fraction of Lgr5+ cells relative to other cell types; spatial distribution of cells (e.g. distribution of cells in an organoid such as distribution of: stem cells at the ends of crypt buds; Paneth cells immediately adjacent to Lgr5+ stem cells; and differentiated cells clustered near the central cystic area of an organoid); and combinations of these.

In some embodiments, diagnostic kit 81 is configured to perform one or more tests to determine successful modification of cells, such as is described herebelow in reference to STEP 1070 of FIG. 2. For example, diagnostic kit 81 can comprise components configured to perform a test selected from the group consisting of: PCR-based test; reporter proteins (e.g. eGFP) test; his-tagging test (e.g. of a reporter protein and/or of an otherwise functional protein of interest); antibiotic selection test (e.g. a test for resistance to puromycin); a test for modified surface/transmembrane proteins; and combinations of these.

In some embodiments, diagnostic kit 81 is configured to perform one or more tests on byproducts produced during the creation of material 60, such as to avoid losing a portion of material 60 to testing. For example, diagnostic kit 81 can include components configured to perform an FACS analysis on trypsinized waste tissue, such as to determine a ratio of K cells to L cells or ratio of GIP producing cells to GLP-1 producing cells, such as to confirm identity of the donor.

In some embodiments, diagnostic kit 81 is configured to perform a test to determine safety and/or efficacy of material 60 prior to implantation into patient P1, such as is described herebelow in reference to STEP 1115 of FIG. 2. For example, diagnostic kit 81 can comprise components configured to confirm a parameter level selected from the group consisting of: endotoxin and/or other toxin levels are below a threshold; bioburden is below a threshold; mycoplasma level below a threshold; adventitious agent below a threshold; cell viability above a threshold, such as above a threshold of 70%; percentage of Lgr5+ cells above a threshold, such as above a threshold of 1%, or 2%, or 30%; percentage of Paneth above a threshold, such as above a threshold of 1%, or 2%, or 30%; transduction copies per cell above a threshold; potency above a threshold; and combinations of these. In some embodiments, diagnostic kit 81 can include components configured to assess potency of material 60. For example, the potency assessment can include a quantified expression of incretins and/or an expression ratio of one incretin to another (e.g. expressions that can be compared to a threshold to determine adequacy of material 60). Alternatively or additionally, the assessment can comprise a quantification of the number of cells, crypts, and/or organoids present in the sample. Diagnostic kit 81 can include components configured to provide identity information (e.g. anatomical location information) of the material 60, such as by quantifying and/or detecting markers of source tissue from cells other than enteroendocrine cells. For example, presence of fatty acid binding proteins (that are produced abundantly by enterocytes in the duodenum) would indicate duodenal tissue versus distal intestinal tissue. Diagnostic kit 81 can include components configured to perform a blood test, such as a blood test of patient P2 and/or patient P1. Blood tests of patient P2 can be used to evaluate the patient's suitableness for the procedure. Blood tests that evaluate circulating levels of hormones, presence of particular antibodies, and/or some other blood borne marker can be performed, such as to assess applicability of the patient, to perform dosimetry calculations, and/or to assess projected success of the treatment.

In some embodiments, system 10 includes storage kit 82 which includes one or more components configured to store harvested tissue 61 and/or material 60 (e.g. a partially or completely processed material 60). Storage kit 82 can include components (e.g. reusable components and/or disposable, single-use components) configured to store tissue and/or other material, such as is described herebelow in reference to method 1000 of FIG. 2.

Storage kit 82 can comprise one or more containers configured to store tissue or other material. The containers can comprise one or more locking features, and they can include a unique ID (or accommodate placement of a unique ID) used to provide traceability through its use.

Storage kit 82 can comprise environmentally controlled storage containers, such as containers which control temperature, humidity, pressure, and the like. For example, storage kit 82 can comprise a refrigeration unit which attaches to and/or includes one or more containers, such as to maintain material 60 at a temperature below room temperature. Storage kit 82 can comprise packing and other materials used to ship material 60 from one location to another location, such as from a clinical setting in which tissue 61 is harvested from patient P2 to a processing setting in which material 60 is produced, and/or from a processing setting to a clinical setting in which material 60 is deposited in patient P1.

Storage kit 82 can include one or more cleaning or other agents used to wash tissue 61 and/or material 60 such as is described herebelow in reference to method 1000 of FIG. 2. For example, storage kit 82 can comprise a surfactant and/or a detergent (e.g. Triton X-100 or SDS), and/or simply phosphate-buffered saline used in a washing procedure.

Storage kit 82 can include one or more storage solutions, such as an isotonic balanced salt solution. The storage solution can comprise an antimicrobial agent such as penicillin. The storage solution can comprise a preservative such as a preservative selected from the group consisting of: a preservative configured to arrest cellular apoptosis; a Rho kinase inhibitor (such as Y27632), such as at a concentration of 5 μM to 15 μM; an antimicrobial reagent such as Primocin, such as at a 0.1% to 0.3% v/v solution; dimethyl sulfoxide (DMSO), such as at a 10% v/v solution (e.g. if shipped cryopreserved); and combinations thereof.

In some embodiments, storage kit 82 comprises one or more safety assemblies 83 described herebelow.

In some embodiments, system 10 includes safety assembly 83 which includes one or more components configured to assure the safety and/or efficacy of material 60 prior to its implantation in patient P1. In some embodiments, safety assembly 83 comprises one or more components configured to confirm that tissue 61 and/or material 60 is not exposed to an undesired temperature (e.g. including at a high or low temperature for an undesired amount of time), at an undesired pressure, at an undesired force, at an undesired pH level; or at another undesired physical state, such as when safety assembly 83 comprises one or more temperature, pressure, force, pH, and/or other sensors and safety assembly 83 is configured to accompany tissue 61 during its storage and/or transportation (e.g. travel from one location to another location). For example, safety assembly 83 can comprise a color strip configured to change colors when an undesired condition is met (e.g. an undesired temperature). Safety assembly 83 can comprise a tensile force indicator, such as a string or other filament that is positioned between two containers containing tissue 61 and/or material 60, such that breakage of the filament is indicative of an undesired force having been imparted on the containers.

Safety assembly 83 can comprise one or more components configured to destroy material 60 if an adverse condition is detected (e.g. by one or more sensors of safety assembly 83), such as to absolutely prevent material 60 from being deposited in a patient. For example, safety assembly 83 can include a portion of tissue modification kit 86, the portion including genetic material that can be included (e.g. inserted into) material 60 to cause the death of a cell when undesired (e.g. unsafe) conditions are encountered, such as is described herein.

In some embodiments, system 10 includes an identification kit, ID kit 84 which includes one or more components configured to identify tissue 61 and/or material 60 prior to the deposit of material 60 into a patient. The ID provided by ID kit 84 can be configured to travel with tissue 61 and/or material 60 during its transportation between settings, and/or during processing. ID kit 84 can provide traceability information that is compatible with clinical electronic record systems (e.g. such as through the use of unique barcodes identifying the material and/or the intended patient). ID kit 84 can provide identifiers that are applied to one or more storage containers of storage kit 82 (e.g. lockable storage containers). In some embodiments, ID kit 84 can include identifiers (e.g. serial numbers or other identifiers) for one or more components of system 10 used to create material 60 and/or deposit material 60 in a patient (e.g. identifiers for harvesting device 400, processing device 500, depositing device 600, and/or treatment device 700).

In some embodiments, system 10 includes tissue expansion kit 85, which includes one or more components configured to culture, amplify and/or otherwise expand tissue (e.g. expand the quantity of tissue). Tissue expansion kit 85 can comprise one or more culture containers in which tissue is expanded. Tissue expansion kit 85 can comprise culture containers with basement membrane domes, that can be immersed in growth media within individual wells of a multi-well plate. Tissue expansion kit 85 can comprise culture containers including a layer of basement membrane evenly spread across the bottom of a culture flask (e.g. a T25 flask), covered in growth media. Tissue expansion kit 85 can comprise a culture container including a basement membrane placed on a permeable membrane, such as to allow simplified replacement of culture media. In some embodiments, tissue expansion kit 85 includes spheres comprising a basement membrane matrix, and a culture media filled bioreactor into which the spheres can be suspended (e.g. be free-floating versus adhered to a surface). Tissue expansion kit 85 can further include a fluid handling assembly configured to replace the media as needed and/or to move the spheres (via a carrier fluid) through the bioreactor (e.g. for each cell culturing passage).

In some embodiments, tissue expansion kit 85 comprises equipment and other components that causes organoids to be formed (i.e. material 60 comprises organoids).

Tissue expansion kit 85 can comprise tissue culture growth medium, such as basal medium. In some embodiments, the growth medium comprises: DMEM/F12; 10 mM HEPES; 2 mM GlutaMax; and/or 1% PenStrep. In some embodiments, one or more additives are included, such as an additive selected from the group consisting of one or more of: a trophic factor; a growth factor; a temporary additive; and combinations of these. For example, tissue expansion kit 85 can comprise a growth factor selected from the group consisting of: Gastrin 1, such as at a concentration of 10 nM; N Acetylcysteine (NAC), such as at a concentration of 1 mM; B27 supplement, such as at a concentration of 2% v/v; Wnt3A, such as at a concentration of 100 ng/mL; R-spondin 1, such as at a concentration of 1 μg/mL; Noggin, such as at a concentration of 100 ng/mL; epidermal growth factor (EGF), such as at a concentration of 50 ng/mL; A83-01, such as at a concentration of 500 nM; SB202190 (p38 MAP kinase); and combinations thereof. Tissue expansion kit 85 can comprise a temporary additive, such as Y-27632 (ROCK inhibitor) and/or CHIR99021 (GSK-3 inhibitor), each of which can be added at the onset of culturing and then removed after a limited time period such as 2 days. In some embodiments, tissue expansion kit 85 comprises a putative growth factor including the native molecule and/or analog forms or variants of a substance selected from the group consisting of: insulin; gastrin; betacellulin; amphiregulin; TGF-alpha: transforming growth factor-alpha; epidermal growth factor (EGF); heparin binding epidermal growth factor (HB-EGF); GLP-1: GLP-2; growth hormone; insulin-like growth factor-1 (IGF-1); granulocyte colony stimulating factor (G-CSF); erythropoietin (EPO); intestinal trefoil factor (ITF); keratinocyte growth factor (KGF); hepatocyte growth factor (HGF); neuregulin-4 (NRG-4); and combinations of these.

In some embodiments, addition or subtraction of growth factors is employed for primary versus subcultures. For example, CHIR99021 could be used only to establish primary culture, and then not used after first passage of amplification. Tissue expansion kit 85 can include a selection agent (e.g. puromycin) to eliminate non-transfected cells. Tissue expansion kit 85 can include an essential nutrient, added to the culture media to support cell survival.

Tissue expansion kit 85 can include one or more support structures, such as to support organoid growth of the material 60 expansion process. For example, tissue expansion kit 85 can include a support structure selected from the group consisting of: a basement membrane matrix comprising a gelatinous protein mixture (e.g. Matrigel); basement membrane extracts (BMEs); a PEGylated hydrogel; a hydrogel with tunable elasticity (e.g. that can be configured to provide beneficial effects on cell proliferation and differentiation); and combinations thereof.

In some embodiments, system 10 includes tissue modification kit 86, which includes one or more components configured to modify tissue 61 and/or material 60. In some embodiments, tissue modification kit 86 includes equipment, material, and/or other components configured to genetically modify tissue 61 and/or material 60.

Tissue modification kit 86 can comprise a gene delivery mechanism, such as a mechanism selected from the group consisting of: transposon (e.g. a PiggyBac transposon); viral vector (e.g. retrovirus, lentivirus, adenovirus, adeno-associated virus); CRISPR-Cas9; electroporation and/or sonoporation mechanism; Lipofection; and combinations of these.

Tissue modification kit 86 can be configured to perform a genetic modification selected from the group consisting of: gene “knock-out” (whereby a gene is made inoperative); gene “knock-in” (whereby a gene or portion thereof is substituted for another gene or portion thereof); modification of noncoding portions of the genome (e.g. promoters); insertion of genes (e.g. native or non-native genes), promoters, and/or DNA; and combinations of these. A gene, promoter, and/or DNA could be inserted to augment expression of a particular protein, to reverse expression of a particular protein, to cause expression of a protein (e.g. a protein that is not currently being expressed by the cell either due to genetic mutation or other reason), and/or to prevent expression of a protein.

Tissue modification kit 86 can include a piece of genetic material that can cause the death of the cell when either a specific nutrient is delivered to the cell and/or when the cell is denied a specific nutrient.

In some embodiments, system 10 includes cell sorting kit 87, which includes one or more equipment, materials, and/or other components configured to sort cells of tissue 61 and/or material 60.

Cell sorting kit 87 can be configured to perform a cell sorting process using one or more of: presence of cell surface antigens such as Lgr5 (e.g. coupled with FACS, MACS, and the like); detection of proteins via intracellular staining coupled with flow cytometry (e.g. when only cells with intracellular GIP are selected); selection by application of an antibiotic such as puromycin (e.g. when the transduced cells have antibiotic resistance imparted as part of a genetic modification that has been performed); density gradient separation such as centrifugation (e.g. possibly following a dissociation and/or trypsinization step); and/or filtering through a porous membrane with a particular pore size such as an approximately 70 micron pore size (e.g. possibly following a dissociation and/or trypsinization step).

In some embodiments, system 10 includes a kit for assembling material 60, deposit material assembly kit 88, which includes one or more equipment, materials, containers, and/or other components configured to assemble material 60.

Deposit material assembly kit 88 can include one or more preservatives (e.g. cryopreservative), one or more antibiotics (e.g. penicillin), growth enhancers, growth inhibitors, media, and/or other materials for combining with material 60 (e.g. for storage and/or transportation). If the cells of material 60 have been genetically modified to have an antibiotic resistance, then adding an antibiotic (e.g. puromycin) to material 60 can give the cells of material 60 an inherent growth advantage over the native cells (deposit location cells) that are not resistant to the antibiotic. The concentration of the antibiotic need not kill the native tissue, only retard its regeneration enough that the new tissue (resultant tissue herein) has a growth advantage. In some embodiments, one or more of these antibiotics or other additives are included in material 60. Alternatively, the additive can be introduced at locations proximate the deposit site, in a separate step from delivering material 60 (e.g. prior to, during, and/or after the delivery of material 60 to the deposit site).

In some embodiments, deposit material assembly kit 88 (and/or storage kit 82), includes a container and associated storage medium for storing a portion of material 60, portion 60′, that is not to be deposited in the patient (e.g. not deposited in STEP 1240 described herebelow in reference to FIG. 2). Retention of portion 60′ can be used as a “backup”, in instances where a subsequent depositing of material 60 is desired. This portion 60′ can be processed as described herein, such as an expansion performed via STEP 1060 and/or 1080 that is used to create a sufficient amount of material 60 for depositing.

Referring now to FIG. 2, a flow chart of a method for depositing a material at a deposit site of a patient is illustrated, consistent with the present inventive concepts. Method 1000 comprises a number of steps for harvesting tissue from a mammalian subject at one or more harvest sites, processing the harvested tissue to create one or more materials to be deposited, and depositing the material at one or more deposit sites of a patient. The method of FIG. 2 shall be described using system 10 and its components described hereabove in reference to FIG. 1. The method of FIG. 2 produces material 60 to be deposited at the patient deposit site. As described hereabove, partially processed material 60 shall be referred to as material 60 herein. Resultant tissue that is produced by the depositing of material 60 shall also be referred to as material 60 herein.

In some embodiments, method 1000 of FIG. 2 is performed similar to the methods described in applicant's co-pending application International PCT Patent Application Number PCT/US2019/012338 (Attorney Docket No. 41714-717.601, Client Docket No. MCT-036-PCT), entitled “Material Depositing System for Treating a Patient”, filed Jan. 4, 2019, the contents of which is incorporated herein by reference in its entirety for all purposes.

In STEP 1010, a screening procedure is performed on a mammalian subject, patient P2, such as a screening procedure performed using diagnostic kit 81 such as is described hereabove in reference to FIG. 1. The mammalian subject can be the patient to receive material 60 (i.e. patient P2 and patient P1 are the same subject), and/or it can be a separate mammalian subject. In some embodiments, tissue 61 is harvested from both patient P1 and a separate donor-patient (e.g. patient P2 comprises two mammalian subjects one of which is the patient), such that both patients are screened in Step 1010. In some embodiments, tissue 61 is harvested from a patient P2 comprising two donor-patients that are not patient P1.

In some embodiments, patient P2 is the same mammal as patient P1, and the screening procedure performed in STEP 1010 is similar to the screening requirements described herebelow in reference to STEPs 1200 and/or 1220.

In some embodiments, screening performed in STEP 1010 comprises a diagnostic procedure used to contraindicate patients that have cancer (e.g. cancer of the GI tract), infection (e.g. an infection in the GI tract), presence of C. difficile, HIV, Hepatitis virus A, B, and/or C, syphilis, and/or tuberculosis. In some embodiments, screening performed in STEP 1010 comprises a screening endoscopy, such as an endoscopy to harvest tissue and/or to visualize tissue of the GI tract (e.g. tissue of the harvest site), such as to screen out patients with cancer and/or an infection.

In STEP 1020, a tissue 61 harvesting procedure is performed on patient P2, such as a tissue harvesting procedure performed at one or more harvest sites using harvesting device 400. The tissue 61 harvesting procedure can be performed: via devices inserted through a natural orifice of the patient (e.g. the patient's mouth and/or anus); via devices introduced through the patient's vasculature (e.g. in a transvascular procedure); via minimally invasive surgical tools; and/or in an open surgery.

STEP 1020 can include harvesting tissue 61 at one or more harvest sites, such as one or more harvest sites selected to treat one or more patient medical conditions, such as is described hereabove in reference to FIG. 1. In some embodiments, tissue 61 is harvested from multiple locations, such as from one or more locations of the duodenum, one or more locations of the jejunum, and/or one or more locations of the ileum (e.g. of the terminal ileum).

In some embodiments, the harvesting of tissue 61 can be performed while the patient is in a fasting state (e.g. at least 8 hours after a last meal).

In some embodiments, the harvesting of tissue 61 can be performed after a fast and/or a colonic prep (e.g. when tissue 61 is harvested from the ileum and/or colon of the patient).

In some embodiments, the harvesting performed in STEP 1020 is performed within a maximum time period of performing STEP 1010, such as within 1 year, within 1 month, and/or within 1 week of performing STEP 1010.

In some embodiments, STEP 1020 is performed a minimum time period after the performance of a previous GI procedure. For example, STEP 1020 can be performed at least 30 days after a procedure in which material is deposited in the GI tract (a material 60 implantation procedure as described herein), and/or at least 14 days after a previous GI tissue treatment procedure (e.g. a GI mucosal ablation procedure as described herein).

In some embodiments, STEP 1020 is performed after a minimum time period has elapsed since patient P2 experienced an illness. For example, STEP 1020 can be performed at least 5 days after a GI illness and/or diarrhea is present, and/or at least 90 days after a C. difficile infection is present.

In some embodiments, STEP 1020 is performed after a minimum time period has elapsed since patient P2 has stopped taking a particular medication. For example, STEP 1020 can be performed at least 14 days after patient P2 has finished taking one or more antibiotics.

In some embodiments, STEP 1020 is performed after a minimum time period has elapsed since patient P2 has been ingesting (e.g. relatively continuously ingesting) a particular diet. For example, STEP 1020 can be performed after the patient has been on a gluten-free diet (e.g. a Celiac patient) for at least 30 days, and/or after the patient has been on a low-sugar diet (e.g. a post-bariatric hypoglycemia patient) for at least 30 days.

In some embodiments, STEP 1020 comprises harvesting a minimum number of one or more types of cells. For example, STEP 1020 can comprise harvesting a minimum amount of tissue 61, such as a minimum amount selected from the group consisting of: at least 1 Lgr5+ stem cell of the intestine or other GI tract location; at least lmm³ of tissue (e.g. mucosal tissue); at least 8 mm³ of tissue (e.g. mucosal tissue); and combinations of these. In some embodiments, tissue 61 is harvested via at least 2 or at least 3 biopsies (e.g. mucosal biopsies), such as when different (subsequent) processing occurs with each individual biopsy sample obtained (e.g. different cell amplification, modification, and/or other processing as described in one or more STEPs herebelow).

In some embodiments, tissue 61 is sampled in STEP 1020 via a harvesting device 400 that is advanced from the submucosa to the mucosa, without exiting into the lumen of the GI tract (e.g. without exiting into the lumen of the small intestine as described herebelow in reference to FIGS. 3A-E), to avoid or at least minimize the amount of undesired material (e.g. material present on the wall of the GI tract) being included in the harvested sample.

In some embodiments, STEP 1020 includes a mucosal lift procedure (e.g. a submucosal tissue expansion procedure as described herein) performed prior to the harvesting of tissue 61, such as a mucosal lift procedure performed using harvesting device 400 or another component of system 10. In these embodiments, a mucosal lift can be performed using a vacuum-assisted device. Mucosal lift can be performed via injection of at least 0.25 mL, or at least 0.5 mL, or at least 1 mL of a fluid such as saline (e.g. injection into the submucosa). In some embodiments, a mucosal lift is performed with an injectate (e.g. injectate 2101 described herein) that is visualizable (e.g. an injectate that includes a visualizable dye such as methylene blue). The visualizable material can be used to confirm that a sufficient sample has been obtained (e.g. a sample from the inner wall of the mucosal layer through to the submucosal layer).

In some embodiments, STEP 1020 includes a test of the harvested tissue 61, such as a test performed using diagnostic kit 81. Testing can be performed to confirm: the absence of infected tissue in tissue 61; the absence of cancer tissue in tissue 61; and/or the absence of other undesired tissue and/or undesired material in the harvested sample.

In STEP 1030, tissue 61 is stored, such as storage in a container of storage kit 82 described hereabove in reference to FIG. 1. Tissue 61 can be stored in one or more containers. In some embodiments, storage kit 82 includes a biopsy cassette and storage solution used to store tissue 61.

STEP 1030 can include one or more tests performed on tissue 61, such as are described hereabove in reference to STEP 1020. STEP 1030 can include one or more preparations of tissue 61 prior to storage, such as a washing procedure performed on tissue 61.

STEP 1030 can include the washing of tissue 61, such as a washing using a cleansing agent and/or antimicrobial agent included in storage kit 82.

STEP 1030 can include the combining of tissue 61 with a liquid (e.g. a storage solution), one or more agents, and/or one or more other additives. Tissue 61 can be combined with an additive described hereabove in reference to storage kit 82, such as an additive selected from the group consisting of: a storage solution; an antimicrobial agent; a preservative; and combinations of these.

STEP 1030 can include the storage of tissue 61 at a temperature less than 37° C., such as at a temperature less than 10° C., and/or a temperature above −80° C.

STEP 1030 can include the storage of tissue 61 for no more than 3 months, and/or no more than 7 days (e.g. storage prior to the performance of STEP 1040).

STEP 1030 can include the storage of tissue 61 in two or more separate containers, such as two or more separate containers stored at one, two, or more separate locations.

For purposes of clarity, references to tissue 61 and any additives or other included materials, and references to material 60 and any additives or other included materials, shall be referred to as material 60 in subsequent steps.

In STEP 1040, material 60 is transferred to a setting in which tissue processing is performed.

In some embodiments, transfer of material 60 occurs over a period of time that is no more than 72 hours. Transfer of material 60 can be performed using safety assembly 83, such as is described hereabove in reference to FIG. 1. Safety assembly 83 can be configured to confirm that material 60 has not been adversely affected during transportation.

In some embodiments, transfer of material 60 occurs in a container with a controlled environment, such as a refrigerated container of storage kit 82 as described hereabove.

In STEP 1050, tissue 61 is received at a processing setting (e.g. a tissue processing service company), and tissue processing is performed to create material 60 (e.g. an unfinished, partially-processed version of material 60).

STEP 1050 can include a thawing step, such as when material 60 is transported in STEP 1040 in a frozen or otherwise refrigerated state. Thawing can comprise immersion of material 60 (e.g. a storage container in which material 60 is stored) in a warm water bath.

In some embodiments, STEP 1050 includes a decontamination step, such as when material 60 is washed using one or more cleansing agents and/or antimicrobial agents of storage kit 82 described hereabove.

In some embodiments, STEP 1050 includes a tissue test, as described herein, and/or other diagnostic test, such as a test performed using diagnostic kit 81 described hereabove.

In some embodiments, STEP 1050 is performed in a time period of no more than 6 hours.

Method 1000 of FIG. 2 can include one or more tissue expansion procedures (e.g. tissue culturing and/or amplification procedures), such as those performed in STEPs 1060 and/or 1080. In some embodiments, STEP 1060 is performed and STEP 1080 is not performed (i.e. expansion of material 60 occurs prior to the modifications performed in STEP 1070 but not after). In some embodiments, STEP 1080 is performed and STEP 1060 is not performed (i.e. expansion of material 60 occurs after the modifications performed in STEP 1070 but not before). In some embodiments, STEP 1060 is performed and STEP 1080 is performed (i.e. expansion of material 60 occurs prior to the modifications performed in STEP 1070 as well as after).

The expansion of material 60 in STEP 1060 and/or STEP 1080 (“STEP 1060/80” herein) can comprise a tissue culturing process performed in one or more media provided by tissue expansion kit 85, such as is described hereabove in reference to FIG. 1. One or more additives provided in tissue expansion kit 85, also as described in reference to FIG. 1, can be added to the culture media. Expansion of material 60 can occur over a period of at least 7 days, at least 14 days, and/or at least 28 days. In some embodiments, the media in which material 60 is cultured is replaced on a routine basis, such as at least every 5 days, or at least every 2 days. In some embodiments, tissue expansion kit 85 includes a ROCK inhibitor and/or GSK-3 inhibitors (such as is described hereabove), that are removed after the initial 2 days of expansion (e.g. removed after primary culture plating or passaging).

The expansion of material 60 in STEP 1060 and/or STEP 1080 can include the addition and/or subtraction of certain growth factors for primary passage versus subsequent passages (or subcultures). For example, a particular agent (e.g. CHIR99021) can be used only to establish a primary culture (e.g. not used after a first passage).

In some embodiments, material 60 is split into two portions which are expanded separately from one another, such as to create a redundancy if one portion is damaged or otherwise determined to be unsatisfactory.

The expansion of material 60 in STEP 1060 and/or STEP 1080 can be configured to produce organoids (such as when tissue expansion kit 85 provides components that generate culture conditions that cause organoids to be produced). The organoids can be produced using a supporting structure, such as a basement membrane included in tissue expansion kit 85, such as is described hereabove in reference to FIG. 1.

The expansion of material 60 performed in STEP 1080 can comprise a controlled number of culturing “passages”. In some embodiments, a single passage is performed, and in other embodiments, multiple passages are performed, such as at least 5 passages, at least 10 passages, or at least 25 passages.

STEP 1060 and/or STEP 1080 can include a tissue test performed on material 60, a test that can be performed prior to, during, and/or after expansion of material 60. For example, diagnostic kit 81 can include one or more components (e.g. equipment, materials, and/or other components) to be used to perform a tissue test such as a test selected from the group consisting of: a test for infection; a test for mycoplasma and/or virus; a DNA test (e.g. to ensure accuracy of donor identity); an organoid growth rate test; a test used to identify presence (or lack thereof) of proteins of interest (e.g. GIP, GLP-1, and/or other incretins, such as a test including a response to a glucose-based stimulus); and combinations thereof. Testing performed using diagnostic kit 81 can include testing to assess culture growth rates and/or testing of culture media secretions (e.g. GIP, GLP-1, insulin or other marker peptide not normally secreted by this cell type etc.), each as described hereabove in reference to FIG. 1.

In STEP 1060, expansion of material 60 is performed, such as in a tissue culturing process performed in a culture container included in tissue expansion kit 85, such as is described hereabove in reference to FIG. 1. The expansion of material 60 performed in STEP 1060 can be similar or dissimilar to the expansion in STEP 1080 described herebelow (e.g. it can include similar processes and/or utilize similar components of tissue expansion kit 85 to those described herebelow in reference to STEP 1080).

In STEP 1070, cells of material 60 are modified, such as a genetic or other modification performed using tissue modification kit 86, such as is described hereabove in reference to FIG. 1.

In some embodiments, a genetic modification is performed using a mobile genetic element described hereabove in reference to tissue modification kit 86.

In some embodiments, a genetic modification performed using tissue modification kit 86 comprises a modification selected from the group consisting of: gene “knock-out”; gene “knock-in”; modification of a noncoding portion of the genome (e.g. promoters); insertion of genes (e.g. non-native genes), promoters, and/or DNA; and combinations thereof. In some embodiments, a non-native gene (i.e. a gene that is not naturally present in the cell) is inserted that expresses Exendin-4. In some embodiments, a gene is inserted that is native (i.e. is naturally present) but the native gene is being expressed insufficiently or otherwise expressed incorrectly.

In some embodiments, one or more tests are performed during STEP 1070, such as one or more tests performed using diagnostic kit 81 to confirm successful modification of cells of material 60.

In some embodiments, the processes performed in STEP 1070 include a component of safety assembly 83 that is used to generate an alert and/or destroy material 60 if an undesired modification of material 60 occurs (e.g. an undesired genetic modification is detected). For example, safety assembly 83 and/or tissue modification kit 86 can include genetic material that is included in material 60, the genetic material configured to cause the death of a cell when undesired (e.g. unsafe) conditions are encountered, such as is described herein.

In STEP 1080, an expansion of material 60 is performed, such as in a tissue culturing process performed in a culture container included in tissue expansion kit 85 described hereabove in reference to FIG. 1. The expansion of material 60 in STEP 1080 can be similar or dissimilar to the expansion described hereabove in reference to STEP 1060 (e.g. it can include similar processes and/or utilize similar components of tissue expansion kit 85 to those described hereabove in reference to STEP 1060).

As described hereabove, in some embodiments the expansion of STEP 1060 is avoided, and only the expansion of STEP 1080 is performed (e.g. material 60 is not expanded prior to the modification of STEP 1070).

In STEP 1090, a sorting step is performed on material 60, such as a cell sorting process utilizing cell sorting kit 87, such as is described hereabove in reference to FIG. 1.

The cell sorting performed in STEP 1090 can include sorting based on: detection of a substance; selection by application of an antibiotic; a density gradient-based separation; and/or a filtering process.

In STEP 1100, a check for adequacy of material 60 is performed (e.g. to determine an adequate quantity of one or more types of cells is present), such as a check for adequacy performed using diagnostic kit 81. If it is determined that adequacy is not present, Step 1080 is repeated (e.g. in a cyclic manner with the sorting of STEP 1090 until adequacy is achieved). If adequacy is determined, STEP 1110 is performed.

In some embodiments, STEP 1100 confirms presence of: at least 100 crypts; at least 10,000 crypts; at least 100,000 crypts; at least 1,000,000 crypts; at least 1,000 Lgr5+ cells; at least 100,000 Lgr5+ cells; at least 1,000,000 Lgr5+ cells; and/or at least 10,000,000 Lgr5+ cells, in material 60. If one or more of these minimum quantities are not met, additional material 60 can be produced, such as in the steps described hereabove.

In some embodiments, STEP 1100 includes a cell counting process.

In STEP 1110, material 60 is assembled, and subsequently both steps 1120 and 1115 are performed. The assembly of material 60 performed in STEP 1110 can utilize deposit material assembly kit 88, such as is described hereabove in reference to FIG. 1. STEP 1110 can include separating out portions of material 60 (e.g. separating out organoids, crypts, and/or cells) to be delivered into patient P1. Material 60 (e.g. the resultant material 60 after such a separation process) can be positioned in a suspension or delivery substrate, such as when combined with PBS and/or DMEM (e.g. exclusive of growth factors).

In some embodiments, the assembly performed in STEP 1110 includes adding one or more additives to material 60 (e.g. similar or dissimilar additives to any already added in previous steps). STEP 1110 can include providing a basement membrane to material 60 (e.g. a similar or dissimilar basement membrane to any already present). In some embodiments, material 60 is to be cryopreserved for subsequent shipment, and STEP 1110 includes adding DMSO or an equivalent to support the cryopreservation.

In some embodiments, the assembly of material 60 performed in STEP 1110 includes the removal (including at least reduction in the quantity of, such as a significant reduction) of one or more materials currently present in material 60 (e.g. removal of materials other than the cells, organoids, and/or other material intended to be deposited in patient P1 and/or to support subsequent processing of material 60 up to the time of deposition). For example, growth factors (e.g. that affect cell proliferation and/or differentiation) can be removed from material 60 in STEP 1110 (e.g. removal of these growth factors from the culture material). Removal of material in STEP 1110 can be performed using one or more of: filtering; dilution; precipitation; and combinations of these.

In STEP 1115, a sample of the material 60 produced in STEP 1110 is evaluated (e.g. shipped to an outside lab for evaluation), such as an evaluation using diagnostic kit 81, such as is described hereabove in reference to FIG. 1.

In some embodiments, multiple samples of material 60 are evaluated (e.g. independently tested).

In STEP 1120, the remainder of material 60 is stored, such as storage using one or more containers, storage solutions, and/or other components of storage kit 82 described hereabove in reference to FIG. 1.

In STEP 1130, a check for lot release (release of material 60 produced in STEP 1110) is confirmed (e.g. the evaluation performed in STEP 1115 indicated all criteria tested were at a level acceptable for implantation of material 60 into patient P1). If unacceptable results are determined, material 60 is not deposited in Patient P1 (e.g. the remaining steps are not performed and/or material 60 is destroyed). If acceptable results are determined, STEP 1140 is performed.

In embodiments in which multiple samples of material 60 are evaluated in STEP 1115, acceptable results of testing for all of the samples can be required in order to proceed to STEP 1040.

If acceptable results are determined in STEP 1130, STEP 1140 is performed where material 60 is transferred to a clinical setting (e.g. a setting in which material 60 is to be implanted in patient P1), such as when material 60 is stored in one or more containers, solutions, and/or other components of storage kit 82 described hereabove in reference to FIG. 1.

In some embodiments, one or more of STEPS 1140, 1150, and/or 1200 are performed while the evaluation of material 60 is in-process, however material 60 is prevented from being implanted in patient P1 until a successful lot release is achieved via STEP 1130.

In STEP 1150, material 60 is received and processed.

In STEP 1200, patient P1 is selected and screened, such as is described hereabove in reference to FIG. 1. In some embodiments, the screening involved in STEP 1200 includes the use of one or more components of diagnostic kit 81. The screening can involve determining certain patient information (e.g. patient physiologic information determined using diagnostic kit 81 in one or more patient diagnostic tests) and comparing the determined information to associated inclusion and/or exclusion criteria. These inclusion and/or exclusion criteria can be based on the patient medical condition (e.g. disease or disorder) being treated.

In STEP 1210, patient P1 is sedated.

In STEP 1220, additional patient P1 screening can be performed, such as screening desirably performed under patient sedation and utilizing one or more components of diagnostic kit 81.

In STEP 1230, an optional step of performing a tissue treatment can be performed at or near the deposit site of patient P1, such as a tissue treatment performed using treatment device 700 such as is described hereabove in reference to FIG. 1. The tissue treatment can be performed: via devices inserted through a natural orifice of the patient (e.g. the patient's mouth and/or anus); via devices introduced through the patient's vasculature (e.g. in a transvascular procedure); via minimally invasive surgical tools; and/or in an open surgery. The tissue treatment can comprise a procedure which removes, ablates, denatures, and/or otherwise makes ineffective one or more portions of mucosal tissue proximate each deposit site, also as described hereabove in reference to FIG. 1.

In some embodiments, STEP 1230 comprises a first procedure including a tissue expansion procedure (e.g. an expansion of submucosal tissue in the duodenum or other intestinal location by delivery of injectate 1201 described herein) that is performed prior (e.g. just prior) to a second procedure that includes a tissue ablation or other tissue treatment procedure that makes the treated tissue ineffective, as described herein. In these embodiments, the first procedure can comprise two sequential tissue expansion procedures, performed at two neighboring (e.g. relatively adjacent) intestinal locations.

In STEP 1240, the depositing of material 60 at the deposit site is performed, such as a depositing of material 60 performed by depositing device 600, such as is described hereabove in reference to FIG. 1. The depositing of material 60 can be performed: via devices inserted through a natural orifice of the patient (e.g. the patient's mouth and/or anus); via devices introduced through the patient's vasculature (e.g. in a transvascular procedure); via minimally invasive surgical tools; and/or in an open surgery.

In some embodiments, the depositing of material 60 in STEP 1230 includes a tissue expansion procedure that is performed at the deposit site prior to, during, and/or after the depositing of material 60 at the deposit site, such as is described hereabove in reference to FIG. 1.

In some embodiments, the device used to deposit material 60 in STEP 1240 is the same device used to perform the tissue treatment of STEP 1230 (e.g. depositing device 600 and treatment device 700 are the same device), such as is described hereabove in reference to FIG. 1. In these embodiments, the tissue treatment performed in STEP 1230 can include an ablation procedure that generates significant heat (e.g. a hot fluid, RF, laser, and/or ultrasonic ablation) or generates significant cold (e.g. cryogenic ablation), of one or more portions of the treatment/depositing device 600/700. In these embodiments, system 10 can be configured to position material 60 within device 600/700 to avoid material 60 being exposed to the elevated heat or cold temperature of the ablation. For example, device 600/700 can be cooled and/or warmed as appropriate after the ablation but prior to introducing material 60 into device 600/700. Material 60 can be prevented from being introduced into device 600/700, or at least prevented from being introduced into a portion of device that would cause material 60 to be exposed to the significant heat or cold. Once the heat or cold has been removed (e.g. via time or a temperature neutralizing procedure), material 60 can be introduced and subsequently delivered into the deposit site as described herein. In some embodiments, after the significant heat or cold (of one or more portions of device 600/700) has subsided, material 60 is introduced into device 600/700 such that material 60 is proximate depositing elements 650. In this material 60 introduction, within device 600/700 that is distal to material 60, will exit depositing elements 650. In some embodiments, during the material 60 introduction, delivery elements 650 are retracted or otherwise not positioned within tissue, such that this distal fluid enters the lumen in which device 600/700 is inserted, and not the luminal wall tissue surrounding that lumen.

In some embodiments, material 60 is deposited into submucosal tissue (e.g. submucosal tissue which has been recently expanded as described herein), such as submucosal tissue of the small intestine. In some embodiments, material 60 is deposited on the wall of an axial segment of the GI tract (e.g. the wall of an axial segment of the small intestine), such as when material 60 includes an adhesive agent or other carrier element (e.g. carrier 63 described hereabove in reference to FIG. 1) configured to maintain the relative position of material 60 at the deposit site after being deposited.

In some embodiments, the amount of material 60 deposited at one or more deposit sites comprises a minimum quantity of a component of material 60 (e.g. a minimum quantity of one or more types of cells present in the amount of material 60 to be deposited in patient P1). In some embodiments, at least 10,000 crypts; at least 100,000 crypts; at least 1,000,000 crypts; at least 100,000 Lgr5+ cells; at least 1,000,000 Lgr5+ cells; and/or at least 10,000,000 Lgr5+ cells are deposited in patient P1. In some embodiments, the amount of material 60 deposited is configured to result in the presence of: at least 200 crypts/mm² of deposit site surface area; at least 400 crypts/mm² of deposit site surface area; at least 800 crypts/mm² of deposit site surface area; at least 200 Lgr5+ cells/mm² of deposit site surface area; at least 400 Lgr5+ cells/mm² of deposit site surface area; at least 800 Lgr5+ cells/mm² of deposit site surface area and/or at least 2,000 Lgr5+ cells/mm² of deposit site surface area. In some embodiments, material 60 comprises a liquid carrier (e.g. saline, PBS, a solution of cell nutrients such as DMEM/F12, a liquid including a visualizable agent as described herein, and/or other one or more liquids into which cells can be combined), an extracellular matrix (ECM), and organoids. In these embodiments, the ratio of these materials in material 60 can comprise between 25% and 99.99% liquid carrier (e.g. approximately 90% liquid carrier), between 1% and 90% ECM (e.g. approximately 9% ECM), and between 0.001% and 30% organoids (e.g. approximately 1% organoids). In some embodiments, material 60 comprises a liquid carrier comprising the extracellular matrix, and organoids. In these embodiments, the ratio of these materials in material 60 can comprise between 70% and 99.99% liquid carrier (e.g. approximately 99% liquid carrier), and between 0.001% and 30% organoids (e.g. approximately 1% organoids).

In some embodiments, material 60 is delivered (e.g. using depositing device 600) via one or more fluid delivery elements, at one or more anatomical locations. In these embodiments, each delivery of material 60 (i.e. by a single delivery element at a single anatomical location) can comprise a minimum volume of material 60 (e.g. a volume including cells, organoids, and/or carrier fluid), such as a volume of at least 1 mL, at least 3 mL, at least 5 mL, at least 10 ml, or at least 15 ml. In some embodiments, each delivery of material 60 can avoid exceeding a maximum volume of material 60 delivery, such as a maximum volume of no more that 30 ml, no more than 20 ml, and/or no more than 10 ml.

In some embodiments, a portion of material 60 is not deposited in the patient, that portion being retained for a period of time, such as to be used in subsequent testing based on desired, undesired, and/or other results found in the treatment of patient P1.

In some embodiments, depositing device 600 is positioned in a particular gravimetric orientation, such as to advantageously position any sedimentation of material 60 that may generate during the deposition procedure, such as to ensure that the desired components of material 60 (e.g. cells) are delivered into the deposit site. In some embodiments, vibration and/or other motion is applied to depositing device 600 (e.g. via a manipulating assembly of system 10) and/or motion is applied to a portion of depositing device 600 (e.g. via a motion component of device 600), such as to prevent sedimentation of material 60.

In STEP 1250, an evaluation can be performed, such as an evaluation of the deposit site and/or other evaluation of patient P1.

In some embodiments, the evaluation of STEP 1250 comprises an evaluation to determine if material 60 is properly located at the deposit site. For example, material 60 can include an additive that can be visualized by one or more of: a PET Scanner; a CT Scanner; an ultrasonic imager; and/or an X-ray. Alternatively or additionally, an endoscopic evaluation using a camera (e.g. a visible light camera and/or an infrared camera) can be performed. For example, material 60 can be visualizable (e.g. include visualizable organoids and/or a visualizable additive).

In some embodiments, the evaluation of STEP 1250 comprises an evaluation of stool produced by the patient, such as to assess whether material 60 has undesirably passed through the patient's digestive system (e.g. material 60 was not sufficiently deposited and/or did not sufficiently remain at the deposit site).

STEPs 1230 and 1240 can be repeated two or more times, with or without the inclusion of STEP 1250 (e.g. with or without the evaluation performed in STEP 1250). In some embodiments, STEP 1230 is repeated two or more times, after which one or more STEP 1240's are performed. In some embodiments, two or more STEP 1240's are performed (e.g. after one or more performances of STEP 1230). In some embodiments, one or more STEP 1230's are performed, after which one or more STEP 1240's are performed, and the process is repeated one or more times.

In STEP 1260, a post-procedural regimen can be implemented, such as when patient P1 undertakes a particular diet and/or takes one or more particular pharmaceutical drugs or other agents.

Diagnostic kit 81 can include equipment, materials, and/or other components that can be used to perform a diagnostic test in any one or more of Steps 1010 thru 1260. Diagnostic kit 81 can be configured to perform a test of tissue 61 and/or material 60. Diagnostic kit 81 can be configured to perform a test on patient P2 and/or patient P1.

Storage kit 82 can include equipment, materials, and/or other components that can be used to store material (e.g. store tissue 61, material 60, and/or any other substance) in any one or more of Steps 1010 thru 1260. Storage kit 82 can be configured to store tissue 61, material 60, and/or a biological sample of patient P2 and/or patient P1.

Safety assembly 83 can include equipment, materials, and/or other components that can be used to monitor the safety and/or efficacy of any one or more of Steps 1010 thru 1260. For example, safety assembly 83 can be configured to monitor one or more steps or processes, to assure a minimum time is met and/or a maximum time is not exceeded. Safety assembly 83 can include components that monitor an environmental parameter, such as to assure a safety threshold is not exceeded (e.g. a temperature, pressure and/or force is not exceeded).

Identification kit 84 can include equipment, materials, and/or other components that can be used to identify one or more parts or assemblies used in any one or more of Steps 1010 thru 1260. For example, identification kit 84 can be configured to mark and positively identify tissue 61 and/or material 60, such as to associate an item with a patient P2, patient P1, and/or a unique identifier of the item.

In some embodiments, tissue 61 and/or material 60 are maintained in a relatively cold state for one or more portions of one or more of the above steps, such as at a temperature below 37° C., below room temperature, at or below 10° C., and/or at or below 0° C.

Referring now to FIGS. 3A-C, side sectional anatomical views of a method of performing an “inside-out” biopsy are illustrated, consistent with the present inventive concepts. The inside-out biopsy described herebelow comprises harvesting mucosal tissue with an approach coming from within the submucosal layer. In FIG. 3A, a submucosal expansion device, expansion device 2100, is inserted below the mucosal layer of the GI tract, such as into the submucosal region. In some embodiments, harvesting device 400, depositing device 600, and/or treatment device 700, each as described hereabove in reference to FIG. 1, comprise expansion device 2100. Expansion device 2100 can be configured to deliver submucosal tissue expansion media, injectate 2101, to a submucosal layer region of the GI tract, such as to create a submucosal fluid pocket, bleb 2105 as shown in FIG. 3B. Injectate 2101 can comprise a fluid, such as saline. In some embodiments, injectate 2101 can comprise a dye or other additive configured to allow for enhanced visualization of bleb 2105, for example under direct visualization (e.g. via an endoscopic camera). In some embodiments, injectate 2101 can comprise a preservative or other agent configured to increase the viability of the harvested material collected during the biopsy.

In FIG. 3B, a biopsy device 2200 is shown inserted into bleb 2105 (e.g. bleb 2105 provides a working area for the distal end of biopsy device 2200 in which to be maneuvered). In some embodiments, harvesting device 400 described hereabove in reference to FIG. 1 comprises biopsy device 2200. As shown, biopsy device 2200 can be positioned opposing the mucosal layer, such as to biopsy at least a portion of the mucosal layer (e.g. to harvest tissue 61 comprising a portion of GI mucosa) while approaching the mucosal layer from the submucosal layer. Circle Si depicts the approximate boundary of the tissue 61 to be harvested. As shown in FIG. 3C, the inside-out biopsy captures primarily submucosal tissue and mucosal tissue at least partially separated from the mucosal inner surface (e.g. the biopsy minimizes the amount of the innermost layer of the mucosa harvested). In some embodiments, the mucosal surface of the GI tract can be contaminated with bacteria, microbes, or other contaminates present on the surface of the GI lumen. Performing the inside-out biopsy can avoid or at least minimize the amount of undesired contaminates from the intestinal lumen to be collected with the harvested tissue 61. In some embodiments, the inside-out biopsy is performed without creating bleb 2105.

Referring additionally to FIGS. 3D and 3E, two examples of “outside-in” biopsies are shown, consistent with the present inventive concepts. In FIG. 3D, biopsy device 2200 (e.g. harvesting device 400) is shown opposing the mucosal surface from within the lumen, adjacent to bleb 2105 (e.g. bleb 2105 has been previously created, such as described hereabove). In this embodiment, some contaminates may be captured with the harvested material, as depicted by circle Sz. As described hereabove, in some embodiments injectate 2101 can comprise an antibiotic to treat contamination of the harvested tissue. In FIG. 3E, biopsy device 2200 is shown opposing the mucosa from within the lumen, without a bleb or other submucosal tissue expansion. Circle S₃ depicts the mucosal tissue and contaminates harvested with this method. In some embodiments, the harvested tissue is washed and/or otherwise treated, as described herein, to remove or at least reduce presence of contaminates in harvested tissue 61.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. (canceled)

2. A method of treating a medical condition, the method comprising:
harvesting tissue with a harvesting device from a mammalian subject at multiple harvest sites within the ileum and/or the colon;
processing the harvested tissue with a processing device, the processing device comprising a cell sorting kit configured to perform a cell sorting process based on the presence of cell surface antigens; and
depositing material with a depositing device in a patient at a deposit site, the material based on the harvested and processed tissue;
wherein the deposited material is configured to generate resultant tissue configured to treat the medical condition of the patient; and
wherein the medical condition comprises insulin resistance.

3. The method according to claim 2, wherein the method is further configured to treat a medical condition selected from the group consisting of: Type 2 diabetes; non-alcoholic fatty liver disease; obesity; an obesity-related disorder; non-alcoholic steatohepatitis; pre-diabetes; liver insulin resistance; and combinations thereof.

4. The method according to claim 2, wherein the harvesting device is configured to perform a biopsy.

5. The method according to claim 2, wherein the harvesting device is configured to perform an endoscopic mucosal resection procedure and/or an endoscopic submucosal dissection procedure.

6. The method according to claim 2, further comprising performing a first treatment with a first treatment device, the first treatment device configured to ablate and/or remove tissue at or otherwise proximate the deposit site.

7. The method according to claim 6, further comprising performing a second treatment with a second treatment device, the second treatment device configured to perform a tissue expansion procedure at or otherwise proximate the deposit site.

8. The method according to claim 7, wherein the first treatment device and the second treatment device are the same device.

9. The method according to claim 8, wherein the depositing device, the first treatment device, and the second treatment device are the same device.

10. The method according to claim 2, further comprising performing a treatment with a treatment device, the treatment device configured to treat tissue prior to, during, and/or after deposit of the material at the deposit site.

11. The method according to claim 2, further comprising delivering an agent to the patient and/or the mammalian subject.

12. The method according to claim 2, further comprising performing an analysis with a diagnostic kit including one or more components.

13. The method according to claim 12, wherein the diagnostic kit is configured to perform an analysis of the material.

14. The method according to claim 12, wherein the diagnostic kit is configured to assess a concentration and/or ratio of one or more substances of tissue.

15. The method according to claim 12, wherein the diagnostic kit is configured to perform a test to determine safety and/or efficacy of the material prior to deposit in the patient.

16. The method according to claim 12, wherein the diagnostic kit is configured to assess potency of the material.

17. The method according to claim 16, wherein the potency assessment comprises a quantification of the number of cells, crypts, and/or organoids in the material.

18. The method according to claim 2, further comprising storing at least a portion of the tissue and/or material within a storage kit including one or more components.

19. The method according to claim 2, further comprising assuring the safety and/or efficacy of the material prior to its deposit in the patient with a safety assembly including one or more components.

20. The method according to claim 19, wherein the safety assembly is configured to confirm the tissue and/or material has not been exposed to an undesired temperature.

21. The method according to claim 20, wherein the undesired temperature comprises either a high or low temperature for an undesired amount of time.

22. The method according to claim 19, wherein the safety assembly comprises one or more components configured to destroy the material if an adverse condition is detected.

23. The method according to claim 2, wherein the depositing device comprises a device configured to implant, place, seed, inert, spray, and/or topically apply material at the deposit site.

24. The method according to claim 2, wherein the processing device is further configured to tag tissue with a fluorescent or other maker so that the tissue is identifiable after its deposit within the patient.

25. The method according to claim 24, wherein the tagged tissue is used to assess treatment longevity, assess efficacy of the depositing procedure, and/or identify tissue to be removed in a subsequent treatment.

26. The method according to claim 25, wherein tissue is removed if an undesired treatment effect is encountered, and removal of the tissue reverses or reduces the undesired effect.

27. The method according to claim 2, wherein the cell sorting process is based on the presence of Lgr5.
